(19) United States
(12) Patent Application Publication
(10) Pub. No.: US 2023/0320896 A1
(43) Pub. Date: Oct. 12, 2023

(54) HANDS-FREE DEVICES AND METHODS FOR THE PREVENTION AND TREATMENT OF MIGRAINES AND OTHER HEAD AND NECK DISORDERS

(71) Applicant: Jason Michael PARNES, New City, NY (US)

(72) Inventor: Jason Michael PARNES, New City, NY (US)

(21) Appl. No.: 18/129,465

(22) Filed: Mar. 31, 2023

Related U.S. Application Data
(63) Continuation-in-part of application No. 17/822,967, filed on Aug. 29, 2022.
(60) Provisional application No. 63/238,832, filed on Aug. 31, 2021.

(51) Int. Cl. A61F 7/12; A61F 7/00

(52) U.S. Cl. CPC A61F 7/12; A61F 7/0085; A61F 2007/0017; A61F 2007/0075; A61F 2007/0078

(57) ABSTRACT

A hands-free intraoral device for imparting a chilling treatment to a zone of intraoral tenderness within an oral cavity of a user. The hands-free intraoral device includes a carrier having a proximal end and a distal end. The distal end of the carrier is configured to be received within the oral cavity of the user and the proximal end of the carrier is configured to extend to a front and/or externally of the oral cavity of the user. A cooling element formed of a thermally conductive material selected to impart the chilling treatment to the zone of intraoral tenderness. A positioning element is operable to urge at least a portion of the cooling element in abutment with the zone of intraoral tenderness, providing for a hands-free positioning of the cooling element in abutment with the zone of intraoral tenderness.

### CROSS-REFERENCE TO RELATED APPLICATION

This application is a Continuation-in-Part of U.S. patent application Ser. No. 17/822,967, filed Aug. 29, 2022, which application claims the benefit of priority of U.S. provisional application No. 63/238,832 filed Aug. 31, 2021, the contents of each of which are herein incorporated by reference in their entirety.

### BACKGROUND OF THE INVENTION

The present invention relates to intraoral devices, and more particularly to intraoral devices which cool an area of a plexus formed by one or more of a posterior superior or a middle superior alveolar branch of an ipsilateral maxillary nerve, known herein as the zone of intraoral tenderness.

A tension-type headache, migraine, post-traumatic headache, atypical facial pain, cervical muscle hyperactivity, and other conditions are each symptoms of ipsilateral intraoral tenderness, as discovered by Dr. Mark H. Friedman, DDS. As disclosed in U.S. Pat. No. 5,527,351, issued Jun. 18, 1996, U.S. Pat. No. 5,676,691, issued Oct. 14, 1997, and U.S. Patent Application Publication Number U.S. 2007/0032547, published Feb. 8, 2007, Dr. Friedman prevented numerous patients from ever experiencing a migraine and other ailments again, including the Inventor herein, by chilling the zone of intraoral tenderness for forty minute sessions via metal probes applied to the zone and cooling the temperature of the probes to a range of between 0° C.-10° C. In a 2004 publication entitled, “Local Inflammation as a Mediator of Migraine and Tension-Type Headache”, Dr. Friedman analyzed 1,100 migraine patients, 1,026 (93.2%) mostly asymptomatic migraine sufferers who had increased temperature or tenderness, classical signs of inflammation, on the gumline just above the second and third maxillary molars, a zone of intraoral tenderness, which closely related to laterality and severity of reported symptoms. In a clinical trial entitled, “Intraoral Chilling vs. Oral Sumatriptan (Imitrex) for Acute Migraine”, Dr. Friedman showed how intraoral chilling provided significant mean headache relief in pain levels at all time levels measured including the 1, 2, 4, and 24 hour time intervals after the treatment as compared to sumatriptan (Imitrex), with poor relief obtained by placebo. In an ongoing IRB study entitled, “Retrospective Survey of the Effects of Chilling the Zone of Intraoral Tenderness in Patients Experiencing Chronic Migraine and Headache Episodes”, 50% of migraine patients surveyed who underwent an intraoral chilling treatment answered that they were migraine free for 3+ years after their intraoral chilling treatments. 25% of patients reported their migraines did not return. Today, it's been observed that agitating a zone of intraoral tenderness can lead to a headache or migraine within the hour and even up to 24 hours later.

Despite 39 million Americans suffering from the debilitating symptoms of a migraine alone, no invention has been proposed to better chill the zone of intraoral tenderness than the original prototype and its similarly hard to utilize successors, which are collectively over twenty years old. Since Dr. Friedman's passing over a decade ago, no significant resources have been put into addressing or improving his apparatus or his treatment methodology despite their clinical success. Because his devices required a practitioner to properly position and a patient to hold the probes and the number of practitioners utilizing his methods remain limited, there is a need for an improved intraoral device for the treatment of migraines, headaches, and other head and neck disorders and conditions.

Research institutions, pharmaceutical and digital health companies, and charities have been set up to counteract migraines. Despite the billions of dollars in resources invested in the space, no improvements have been made on his clinically successful methodology.

Further, the user's application of Dr. Friedman's probes naturally led users to stick them as far back in the intraoral cavity as possible, due to the shape of the probe and location of the zone. Positioning errors remained due to the user manually holding the probes in position. To withstand the 40 minutes of holding the probes in position, users are encouraged to place a pillow under each arm to steady their arms and hold their hands in position. Then, wrap cloth around the medical tubes as a handle to tolerate the cold. Still, the free-wielding probes led to cooling the wrong area of the intraoral cavity and holding the probes in place for 40 minutes led to muscle fatigue. The difficulty in correctly applying the device and that a dentist needed to apply the treatment led to its lack of public acceptance along with a rise in the popularity of opioids at the time of discovery and far less frequent social media usage by the public.

Further, headache and migraine symptoms specifically are often misdiagnosed by doctors as a possible tumor leading to a costly and potentially dangerous CT Scan or MRI. If no tumor is present, treatment often includes medications, rest, TENs devices, and the avoidance of causal factors including but not limited to certain foods, work, the sun or light, and sound. The pain leaves many with only the hope of prayer because of the debilitating nature of a migraine and lack of reliable treatments available.

Migraine treatment is a specifically crowded space. Current options have a low efficacy rate, are primarily pharmaceutical solutions, and are temporary, meaning at some point in time the user will get another migraine. Despite early success in treating and preventing migraines and other ailments by chilling the zone of intraoral tenderness, none of the current devices or methodologies provide assurance in knowing the hard to reach zone is cooled with a high enough consistency to ensure effective treatment.

It is therefore an object of the present invention to provide more effective treatment solutions for migraines and other ailments mentioned above by chilling the zone of intraoral tenderness.

More particularly, it is an object of the present invention to cool the zone of intraoral tenderness by providing a hands-free device that is more easily applied, yet still more reliably positions the cooling element in abutment with the zone of intraoral tenderness, as further described herein.

Furthermore, it is an object of the current invention that the cooling medium can come in many forms to better cool the hard to access zone of intraoral tenderness.

It is another object of the current invention to provide for more approximate sizing by relying on external factors including but not limited to standing height or jaw size, or a custom mold model allowing users to more reliably point the cooling element towards the zone of intraoral tenderness.

It is another object of the current invention to provide a cooling element extending, protruding, or attached to a device, carrier, clip, or any general widget, allowing users to more reliably direct the cold towards the zone of intraoral tenderness.

### SUMMARY OF THE INVENTION

In one aspect of the present invention, a hands-free intraoral device for imparting a chilling to an area of a plexus formed by one or more of a posterior superior or a middle superior alveolar branch of an ipsilateral maxillary nerve, known herein as the zone of intraoral tenderness, within an oral cavity of a user is disclosed. The hands-free intraoral device includes a carrier having a proximal end and a distal end. The distal end is configured to be received within the back of the oral cavity and the proximal end is configured to extend to a front and/or external of the oral cavity. A cooling element is disposed at the distal end of the carrier. The cooling element is formed of a thermally conductive material selected to impart the chilling to the zone of intraoral tenderness. A conduit communicates a cooling fluid through the hands-free device to the cooling element. A positioning element to position at least a portion of the cooling element in abutment with the zone of intraoral tenderness. The positioning element provides for a hands-free positioning of the cooling element in abutment with the zone of intraoral tenderness.

In other aspects of the invention, a hands-free intraoral device for imparting a heat transfer from the zone of intraoral tenderness within an oral cavity of a user is disclosed. The hands-free intraoral device includes a carrier having a proximal end and a distal end, the distal end is configured to be received within the oral cavity and the proximal end is configured to extend to a front and/or externally from the oral cavity. A cooling element is disposed at the distal end of the carrier. The cooling element is formed of a thermally conductive material selected to receive the heat transfer from the zone of intraoral tenderness to the cooling element. A heat exchanger is configured to transfer the heat from the cooling element. A positioning element to position at least a portion of the cooling element in abutment with the zone of intraoral tenderness. The positioning element provides for a hands-free positioning of the cooling element in abutment with the zone of intraoral tenderness.

In some embodiments, the heat exchanger includes a thermoelectric device. The hands-free intraoral device may also include an electronics package for controlling the thermoelectric device.

In some embodiments, a communications module is configured for a wireless communication with a mobile computing device for controlling the thermoelectric cooler.

These and other features, aspects and advantages of the present invention will become better understood with reference to the following drawings, description and claims.

### BRIEF DESCRIPTION OF THE DRAWINGS

FIG. 1 is a front perspective view of a prong embodiment of the hands-free intraoral device, illustrating a left side prong as used by a user of the device.

FIG. 2 is a top plan view of a prong-embodiment.

FIG. 3 is a frontal view of a user utilizing a prong embodiment of the hands-free intraoral device shown in use for the prevention and treatment of migraines, headaches, and head and neck disorders.

FIG. 4 is a detail top front perspective view and a distal end view of a prong embodiment illustrating angular relationships of a bite pad positioning element with the probe of the hands-free intraoral device, in this case a first angular offset of 15° and a second angular offset of 0°.

FIG. 5 is a detail top front perspective view and a distal end view of a prong embodiment illustrating angular relationships of the bite pad positioning element with the probe of the hands-free intraoral device, in this case a first angular offset of 15° and a second angular offset of +10°.

FIG. 6 is a detail top front perspective view and a distal end view of a prong embodiment illustrating angular relationships of the bite pad positioning element with the probe of the hands-free intraoral device, in this case a first angular offset of 15° and a second angular offset of −10°.

FIG. 7 is a detail top front perspective view and a distal end view of a prong embodiment illustrating angular relationships of the bite pad positioning element with the probe of the hands-free intraoral device, in this case a first angular offset of 10° and a second angular offset of 0°.

FIG. 8 is a detail top front perspective view and a distal end view of a prong embodiment illustrating angular relationships of the bite pad positioning element with the probe of the hands-free intraoral device, in this case a first angular offset of 5° and a second angular offset of 0°.

FIG. 9 is a front perspective view of a user illustrating the general position of the zone of intraoral tenderness.

FIG. 9A is a bottom view of a user's top jaw providing a more detailed illustration of the position of the zone of intraoral tenderness.

FIG. 10 is a front perspective view of a first U-shaped carrier embodiment of the hands-free intraoral device.

FIG. 11 is a front perspective view of a second U-shaped carrier embodiment of the hands-free intraoral device with a resilient urging positioning element.

FIG. 12 is a front perspective view of a third U-shaped carrier embodiment of the hands-free intraoral device with a first loop embodiment of the resilient urging positioning element.

FIG. 13 is a front perspective view of a fourth U-shaped carrier embodiment of the hands-free intraoral device with a flexible arm positioning element.

FIG. 14 a front perspective view of a fifth U-shaped carrier embodiment of the hands-free intraoral device with a cheek fold positioning element.

FIG. 15 is a front perspective view of a sixth U-shaped carrier embodiment of the hands-free intraoral device with a hook positioning element.

FIG. 16 is a front perspective view of a seventh U-shaped carrier embodiment of the hands-free intraoral device with a bite bar positioning element.

FIG. 17 is a front perspective view of an eighth U-shaped carrier embodiment of the hands-free intraoral device with a resilient urging positioning element, a thermoelectric cooler carried on a distal end of the U-shaped carrier, and an electronics package carried on a proximal end of the U-shaped carrier.

FIG. 18 is a front perspective view of the ninth U-shaped carrier embodiment of the hands-free intraoral device with a resilient urging positioning element, a thermoelectric cooler carried on a distal end of the U-shaped carrier, and an electronics package carried on a proximal end of the U-shaped carrier in communication with a mobile computing device.

FIG. 19 is a front perspective view of a tenth U-shaped carrier embodiment of the hands-free intraoral device with a second embodiment of a looped resilient urging positioning element, shown with an optional bite clip.

FIG. 20 is a front perspective view of an eleventh U-shaped carrier embodiment of the hands-free intraoral device with a third embodiment of a looped resilient urging positioning element, and a first lateral tensioner embodiment.

FIG. 21 is a front perspective view of a twelfth U-shaped carrier embodiment of the hands-free intraoral device with a fourth embodiment of a looped resilient urging positioning element and a second lateral tensioner embodiment.

FIG. 22 is a front perspective view of a thirteenth U-shaped carrier embodiment of the hands-free intraoral device with a looped and bite pad positioning element.

FIG. 23 is a front perspective view of the hands-free intraoral device with a with a cheek fold positioning element, with a detail view of an optional multi-lumen conduit.

FIG. 24 is a detail top perspective view of a prong embodiment illustrating a bulbous cooling element and a tooth clip positioning element.

FIG. 25 is a top perspective view of a prong embodiment illustrating a left and a right probe and the tooth clip positioning element.

FIG. 26 is a front perspective view of a prong embodiment illustrating a left and a right probe and the tooth clip positioning element, shown in use treating the zone of intraoral tenderness.

FIG. 27 illustrates a bite knob embodiment of the hands-free intraoral device.

FIG. 28 provides representative dimensions for a probe of the hands-free intraoral device.

FIG. 29 illustrates a clamping embodiment of the hands-free intraoral device.

FIG. 30 illustrates the orientation of the axes of the user.

FIG. 31 is a top perspective view of a prong embodiment including a bite pad positioning element with a vertical teeth guard/guidance plate housing, a diode laser (not shown), a connector in the form of a flexible material attachment (spring) covered by a sleeve and a set of supports, a lower cooling element with a multi-lumen conduit, and a switch for adjusting the flexible material attachment, and thus the cooling element, up and inward or down and outward. FIG. 31A is a back perspective view of the embodiment which includes the diode laser.

FIG. 32 is a top perspective view of a prong embodiment including a connector in the form of a set of supports attached to the vertical teeth guard/guidance plate, which is housing a diode laser (not shown), and the bite pad positioning element. FIG. 32A is a back perspective view of the embodiment which includes a diode laser.

FIG. 33 is a top perspective view of a prong embodiment including a bite pad positioning element with a vertical teeth guard/guidance plate, a connector as flexible material attachment and set of supports, a sleeve covering the flexible material attachment, a switch to manipulate the cooling element up and inwards or down and outwards, and a lower cooling element with a multi-lumen conduit. FIG. 33A is an exploded view of the connector's inner mechanism which is a spring flexible attachment material housed in a sleeve and a set of supports.

FIG. 34 is a top perspective view of a prong embodiment including a connector as a double flexible material attachment with a switch to control the same and a lower cooling element with a multi-lumen conduit. FIG. 34A is an exploded view of the connector's inner mechanism which is a spring flexible attachment material housed in a sleeve, fixed supports housing the outer spring flexible material and the inner spring flexible material attachment which extends from the top of vertical teeth guard/guidance plate which houses supports (not shown).

FIG. 35 is a top perspective view of a prong embodiment having an alternative tooth clip or wedge attachment.

FIG. 36 is a top perspective view of a fourteenth U-shaped mouth guard embodiment with an open front airflow bridge and a high outer rear wall with the cooling element sitting atop the rear outer wall.

FIG. 37 is a top perspective view of a prong embodiment with a bite pad positioning element, a connector as a set of supports, and a vertical teeth guard/guidance plate.

FIG. 38 is a top perspective view of a prong embodiment with a bite pad positioning element, a connector as a set of supports and a lower cooling element with a multi-lumen conduit.

FIG. 39 is a top perspective view of a prong embodiment having a connector in the form of an alternative set of supports and a bite pad positioning element with a vertical teeth guard/guidance plate.

FIG. 40 is a top perspective view of a prong embodiment having a connector as a set of supports, a bite pad positioning element with a vertical teeth guard/guidance plate and a lower cooling element.

FIG. 41 is a top perspective view of a prong embodiment including a bite pad positioning element with a vertical teeth guard/guidance plate, a connector as flexible material attachment and a set of supports, a tensed cooling element pressured by the connector to go upward and inward, a sleeve covering the flexible material attachment, and a lower cooling element. FIG. 41A is an exploded view of the connector's inner mechanism which is a spring flexible attachment material housed in a sleeve and fixed supports.

FIG. 42 is a top perspective view of a prong embodiment including a connector as a double flexible material attachment and a tensed cooling element pressured by the connector to go upward and inward. FIG. 42A is a view of the connector's inner mechanism which is a spring flexible attachment material housed in a sleeve, fixed supports housing the outer spring flexible material and the inner spring flexible material attachment which extends from a set of supports (not shown) inside the vertical teeth guard/guidance plate.

FIG. 43 is a top perspective view of a prong embodiment including a separate diode laser, apart from the vertical teeth guard/guidance plate, but instead attached to the probe.

FIG. 44 is a side plan view of a prong embodiment.

FIG. 45 is a top perspective view of a resiliently urging flexible arm positioning element with one flexible arm.

FIG. 46 is a top perspective view of flexible arms positioning the cooling element in abutment with the zone of intraoral tenderness.

FIG. 47 is a top perspective view of a flexible arm positioning element including a lower (cooling) element or lower cooling element, a flexible arm in the form of flexible attachment material housed in a set of supports. FIG. 47A is a view of the inner mechanism in FIG. 47.

FIG. 48 is a top perspective view of a flexible arm positioning element having a spring flexible material housed in a vertical teeth guard/guidance plate to serve as a flexible arm positioning element. FIG. 48A is a view of the inner mechanism in FIG. 48.

FIG. 49 is a front perspective view of the hands-free intraoral device with a connector attaching a positioning element mold or wedge to the cooling element. FIG. 49A is a front perspective view of a user operably engaging the positioning element.

FIG. 50 is a front perspective view of a hands-free intraoral device with a connector linking a positioning element designed for easy insertion and removal to the cooling element.

FIG. 51 is a front perspective view of a hands-free intraoral device with a positioning element to stabilize the probes and a button to release the tension that is forcing the cooling element upwards and inwards. FIG. 51A is a front perspective view of the hands-free intraoral device operably engaged.

### DETAILED DESCRIPTION

The following detailed description is of the best currently contemplated modes of carrying out exemplary embodiments of the invention. The description is not to be taken in a limiting sense but is made merely for the purpose of illustrating the general principles of the invention. Further, each instance of a hands-free intraoral device, a prong, or the like is intended as one of several different alternative embodiments of the present subject matter.

Broadly, embodiments of the present invention provide a hands-free intraoral device for imparting a chilling treatment to a zone of intraoral tenderness within an oral cavity of a user. The zone of intraoral tenderness is defined by an area of a plexus formed by one or more of a posterior superior or a middle superior alveolar branch of an ipsilateral maxillary nerve, as described in the foregoing patents and patent application publication of Dr. Mark H. Friedman, DDS.

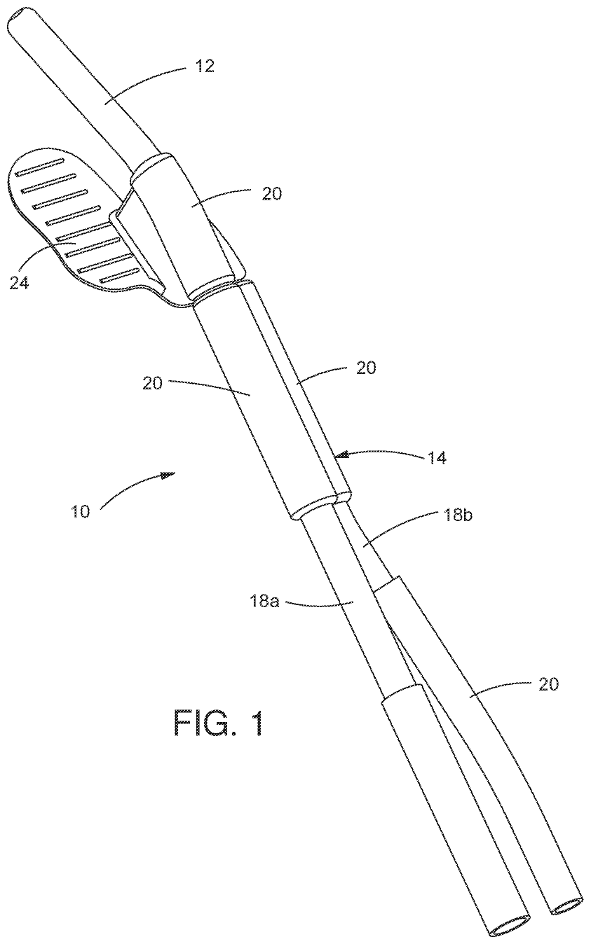

As seen in reference to the drawings of FIGS. 1-4451A the hands-free intraoral device 10 includes a carrier 14 having a proximal end and a distal end. The distal end of the carrier is configured to be received within the oral cavity of the user and the proximal end of the carrier 14 is configured to extend to a front and/or external of the oral cavity of the user.

A cooling element 12 is disposed at the distal end of the carrier 14. The cooling element 12 is formed of a thermally conductive material selected to impart a chilling treatment to the zone of intraoral tenderness. In some embodiments, a cooling fluid is configured to chill the cooling element. A conduit 18 communicates a cooling fluid through the hands-free intraoral device 10 to the cooling element 12. The conduit 18 may include an inflow conduit 18a and an outflow conduit 18b for circulation of a cooling fluid through the conduit 18. The conduit 18 is in a thermally conductive contact with the cooling element 12. The thermally conductive contact may include a circulation of a cooling fluid through the cooling element 12. The conduit 18 is at least partially covered with an insulating material 20 to enhance the chilling effect at the cooling element 12, to prevent the cooling effect from being applied to other areas of the user's oral cavity, and to prevent the cooling element 12 from sticking to the user's gums, though a plastic sheath can be used in some embodiments.

The cooling element 12 should have a smooth outer surface for a thermally conductive contact with the zone of intraoral tenderness and for comfort when positioned within a cheek fold at the juncture of the user's upper jaw line and cheek. The cooling element 12 may also have a bulbous, form-fitting (such as formed by a mold or a gel-like material), or other shape to enhance the thermal contact surface area of the cooling element 12 with the zone of intraoral tenderness.

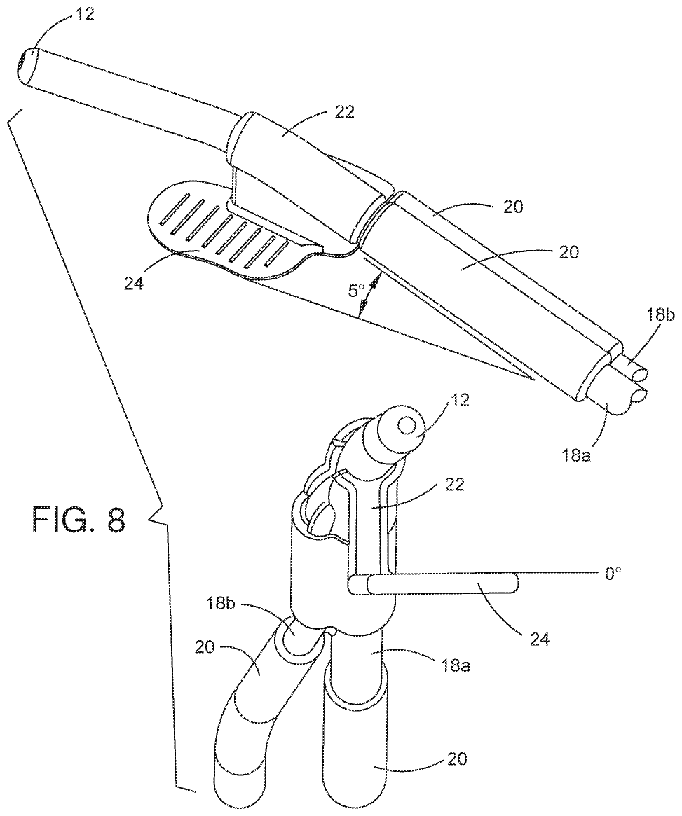

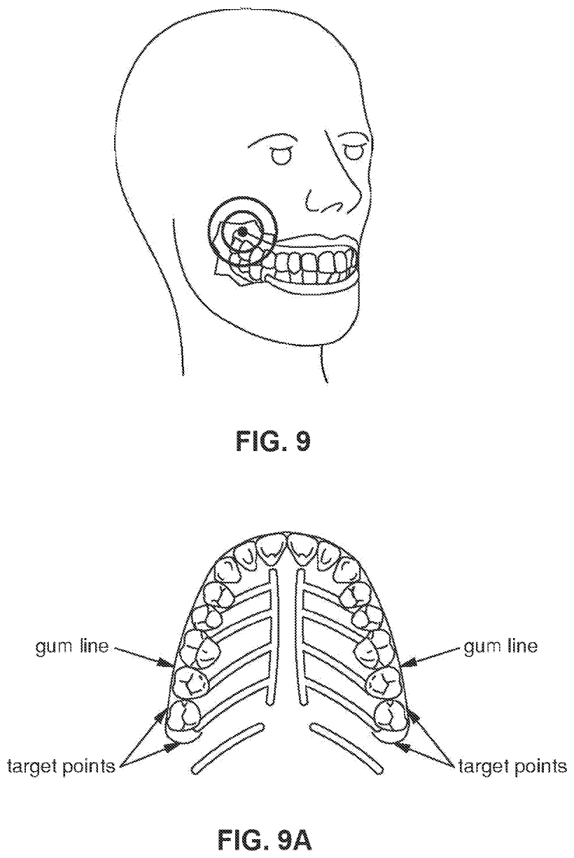

In this regard, FIGS. 9 and 9A show the position of the zone of intraoral tenderness. FIG. 9 shows generally where the zone of intraoral tenderness appears in relation to the rest of the user's face and mouth, while FIG. 9A more specifically shows the location of the zone of intraoral tenderness the cooling element 12 is intended to contact in the far recess of the mouth.

Each embodiment of the hands-free intraoral device 10 includes a positioning element 16 to position at least a portion of the cooling element 12 in abutment with the zone of intraoral tenderness. The positioning element 16 provides for a hands-free positioning of the cooling element 12 in abutment with the zone of intraoral tenderness. The positioning element 16 also facilitates the retention of the hands-free intraoral device 10 in the oral cavity of the user while the user undergoes a cooling treatment session utilizing the hands-free intraoral device 10.

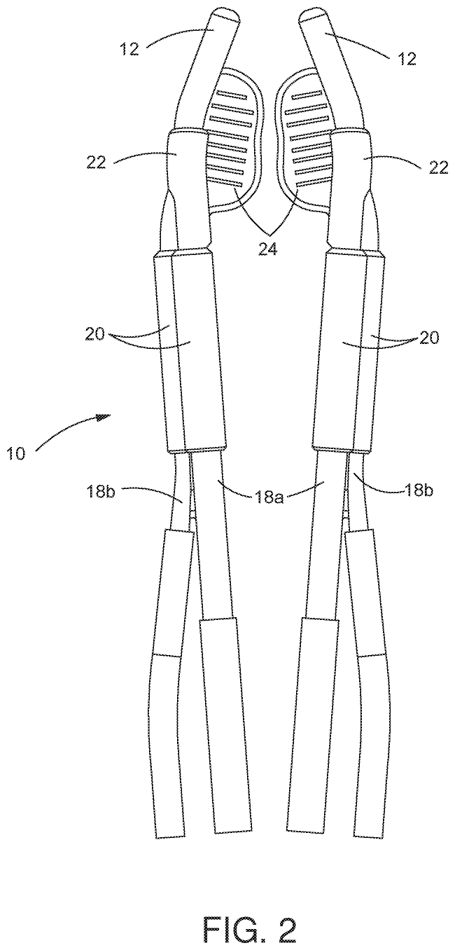

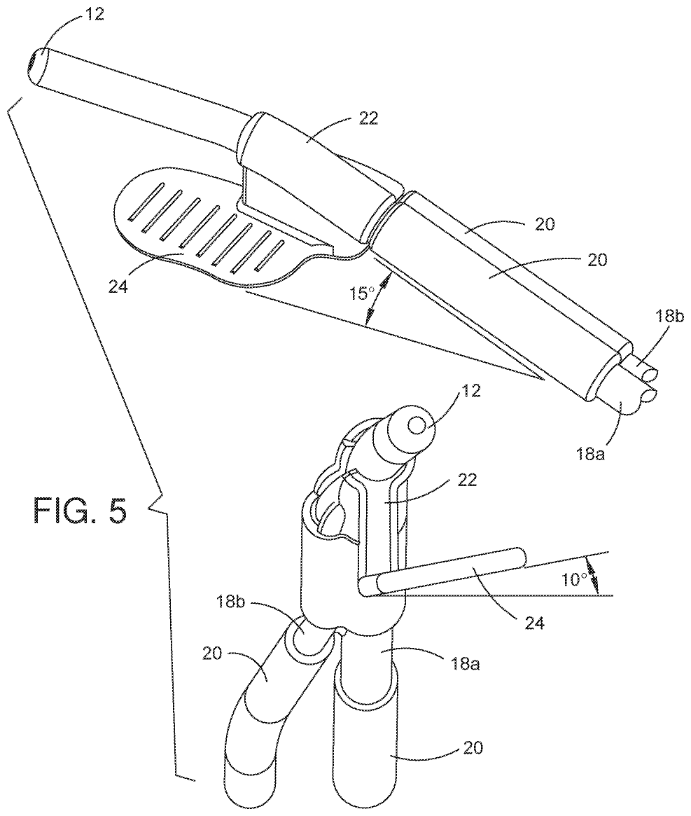

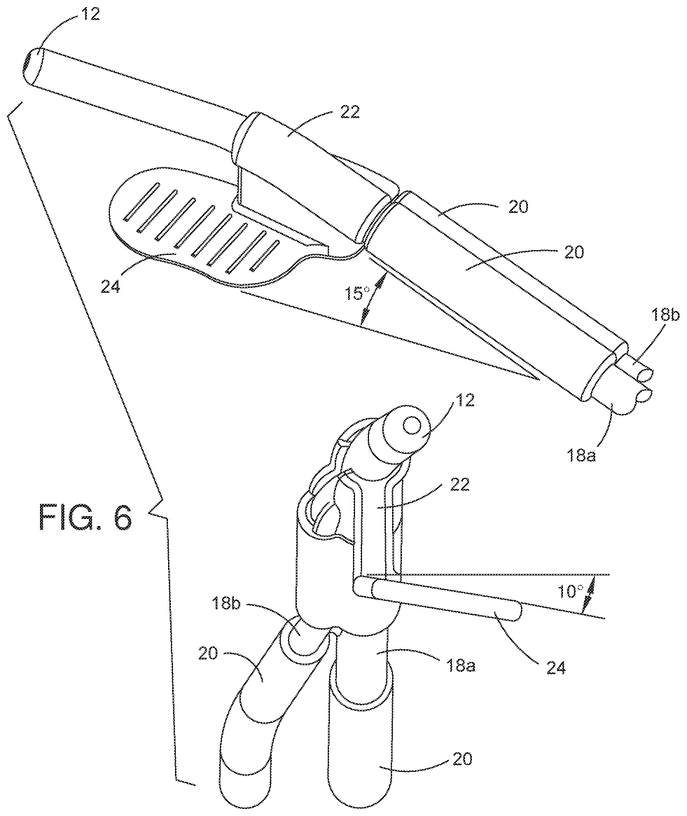

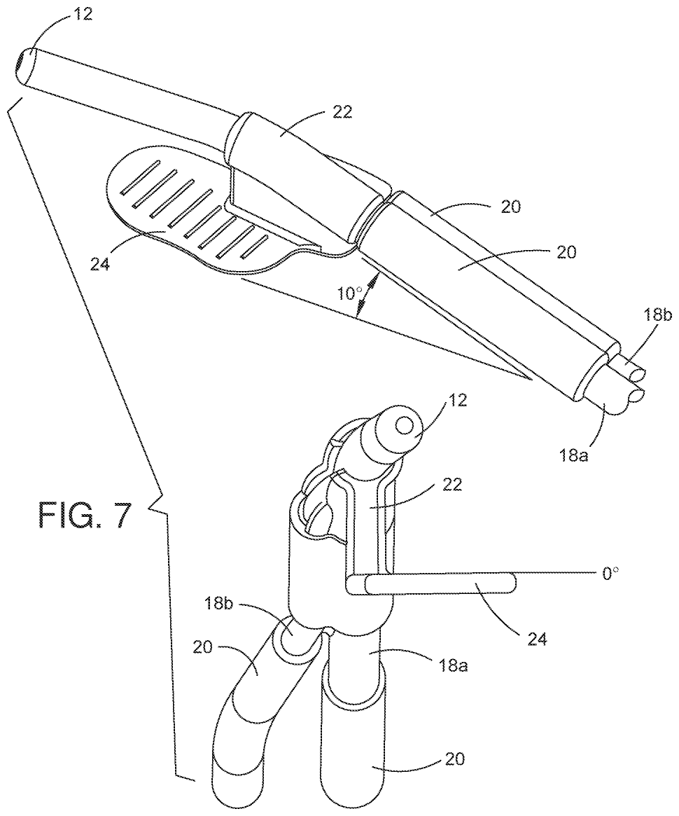

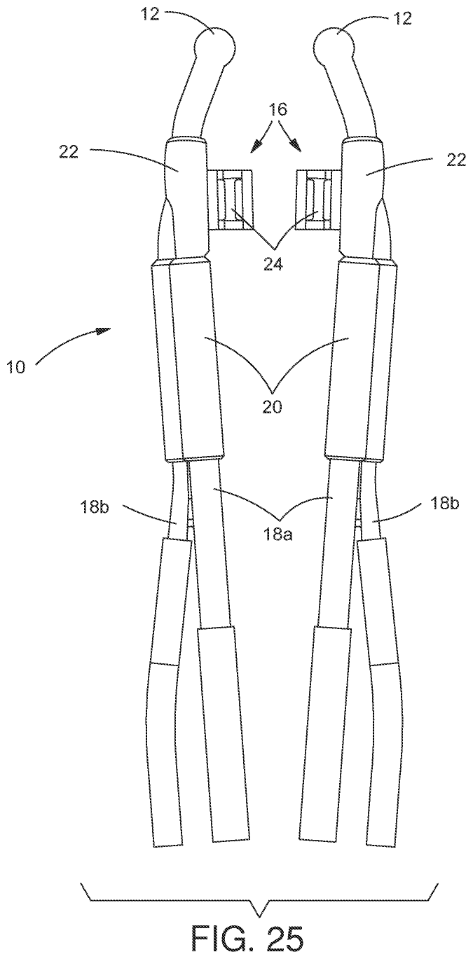

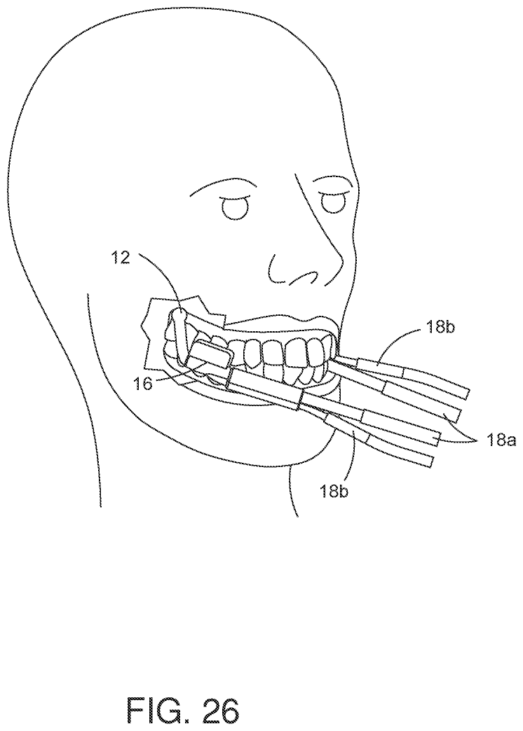

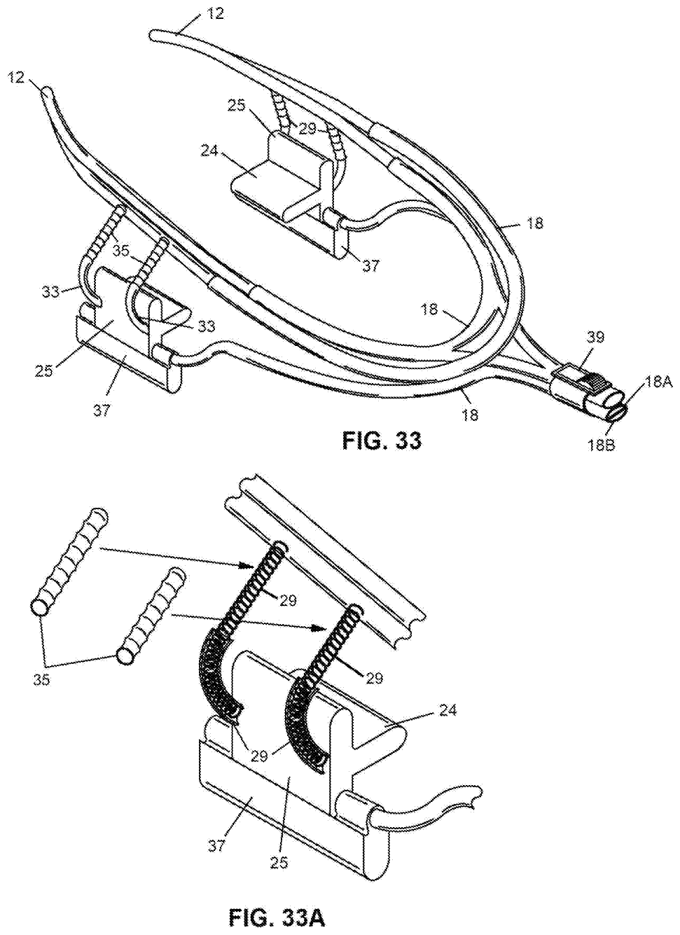

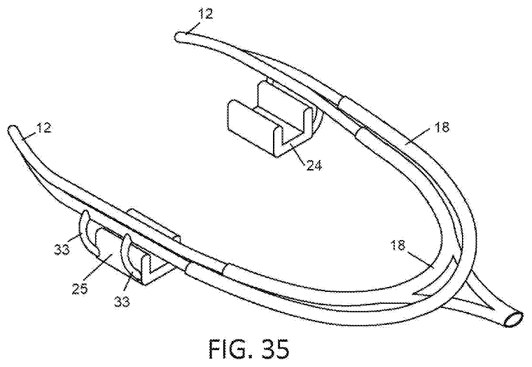

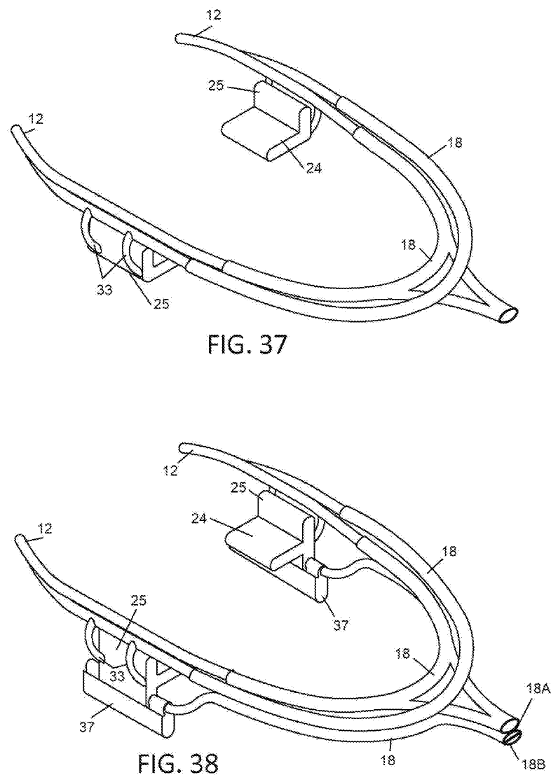

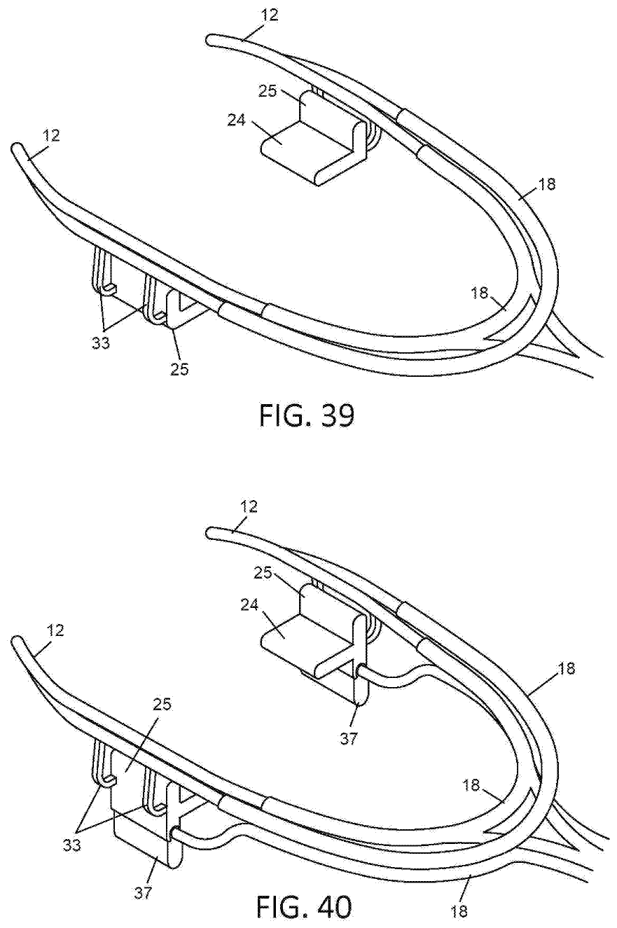

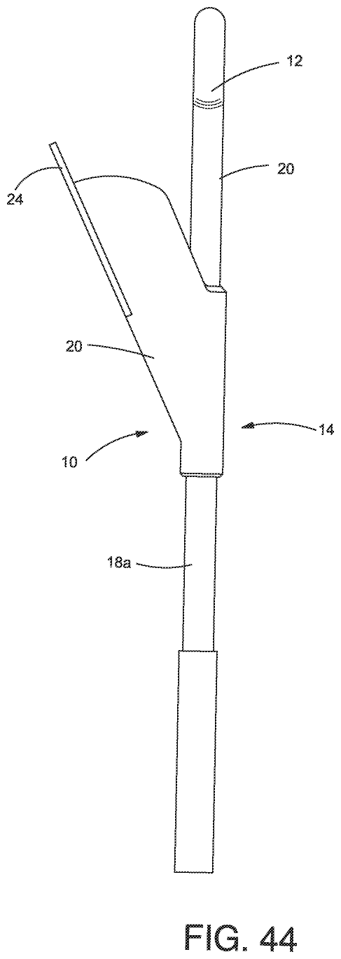

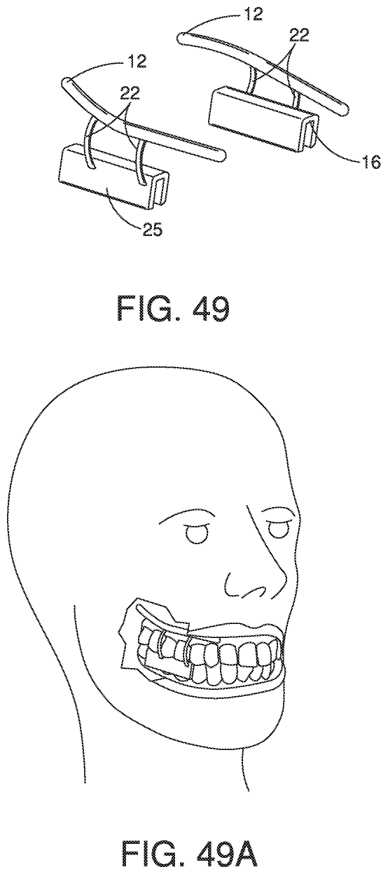

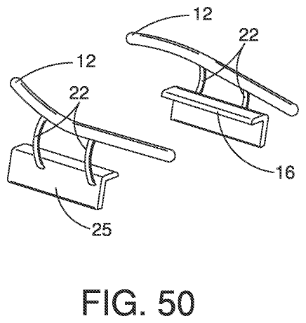

In some embodiments of the hands-free intraoral device 10, such as shown in FIGS. 1-8, 24-26, 31-35, 37-44, and 49-50, the carrier 14 includes a left prong and a right prong. Each of the left prong and the right prong are dimensioned to extend the cooling element 12 into the intraoral cavity to contact the zone of intraoral tenderness.

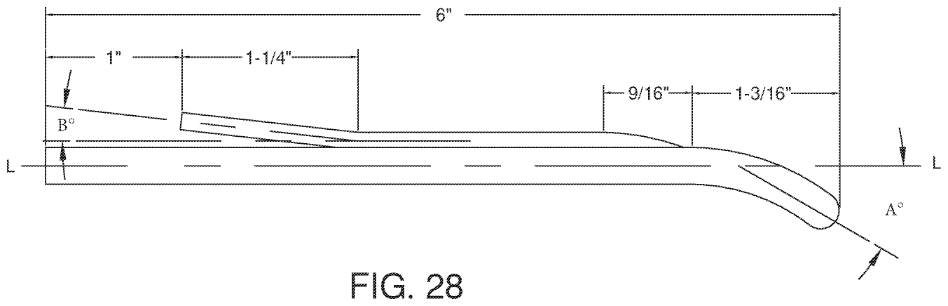

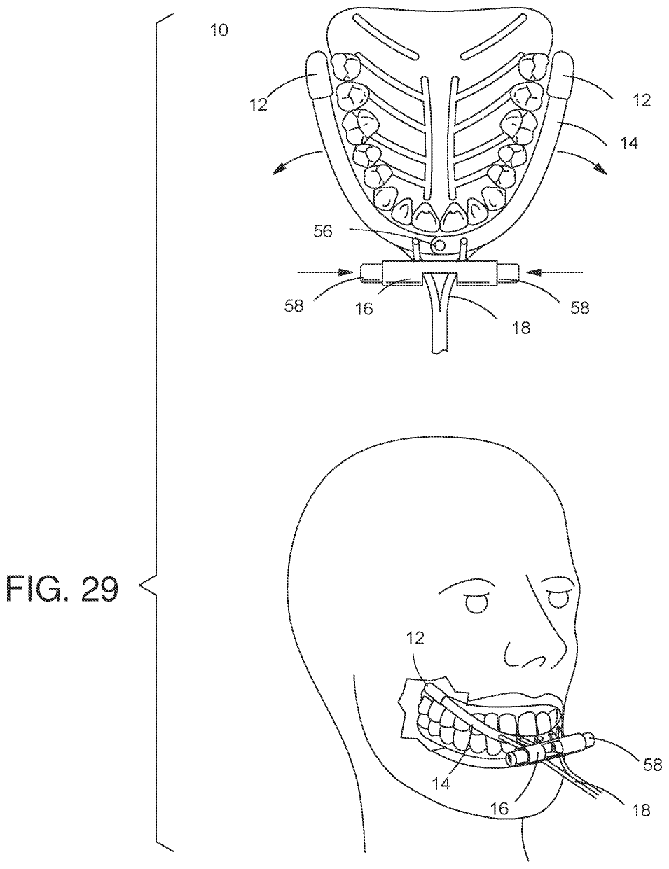

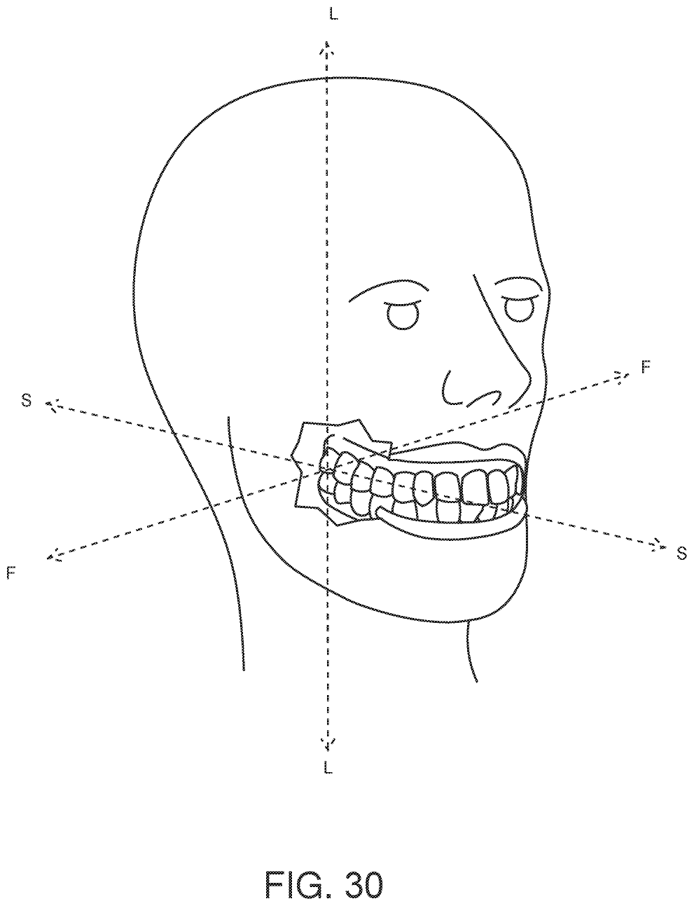

Non-limiting representative dimensions of the probe are shown in FIG. 28. The cooling element 12 is offset from a longitudinal centerline of the carrier 14 by an angle A to allow the cooling element 12 to slide slightly behind the maxillary gumline, the zone of intraoral tenderness. Here, the angle A is approximately 5°. The outflow conduit may be offset by an angle of B° of approximately 6° to provide for connection of a communication tube 18c to couple with the cooling medium (not shown).

In FIGS. 1-8, 24-26, 31-35, 37-44, and 49-50, a connector 22 is at the proximal end of each of the left prong and the right prong. The connector 22 acting as a point or part which the positioning element 16 and the cooling element 12 separately extend, forming each prong. The connector 22 may removably couple the positioning element 16 with the probes to permit replacement of the positioning element 16 for fitting of the hands-free intraoral device 10 to the user and to permit cleaning of the probes between treatments.

A bite pad 24 extends from the connector 22 along a transverse plane towards a sagittal centerline of the intraoral cavity. The bite pad 24 is configured to be operably engaged by a biting of one or more molar teeth of the user to urge the cooling element 12 in abutment with the zone of intraoral tenderness and to retain the hands-free intraoral device 10 within the intraoral cavity. The bite pads 24 and connector 22 may be formed of a rigid, a semi-rigid material, or a resilient material.

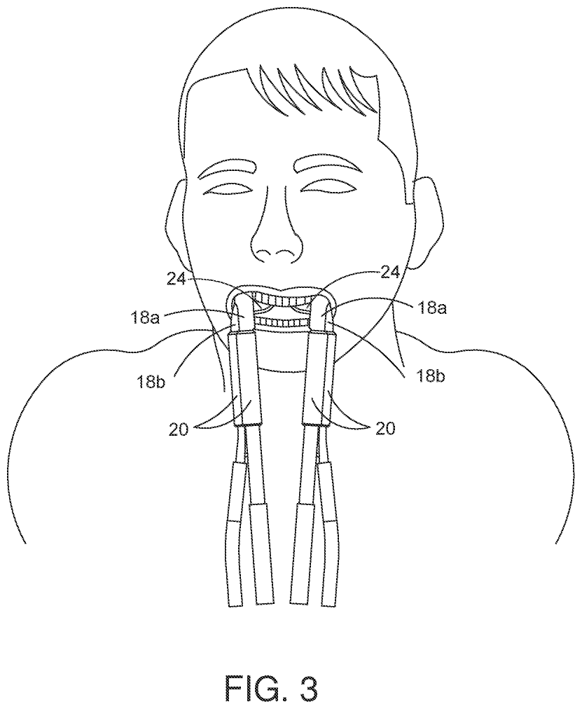

The connector 22 may also be adjustably coupled to provide a positioning adjustment such that the cooling element 12 can be positioned in abutment with the zone of intraoral tenderness. Thus, when the user engages the bite pad 24 between a selected one or more their teeth the cooling element 12 may be reliably positioned within the intraoral cavity to contact the zone of intraoral tenderness. A representative fitting of the hands-free intraoral device 10 is shown in reference to FIG. 3 where the user operably engages the bite pads 24 between their teeth.

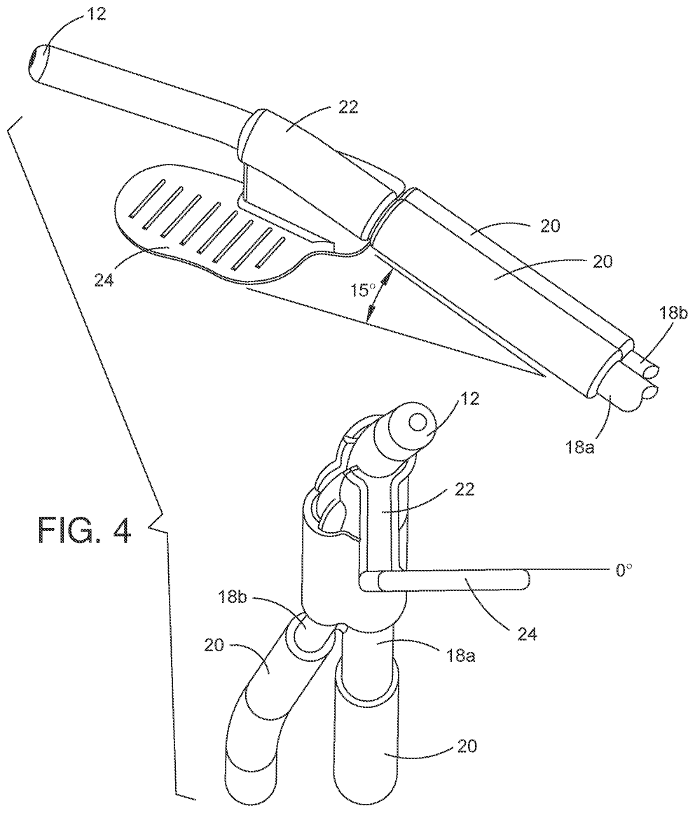

As seen in the embodiment of FIGS. 4-8, for improved fitting of the hands-free intraoral device 10, the bite pads 24 may be oriented at one or more angular offsets so that the cooling element 12 is properly positioned in abutment with the zone of intraoral tenderness and so that the user remains comfortable with the hands-free intraoral device 10 throughout the treatment period.

The one or more angular offsets may include a first angular offset of the bite pad 24 about a frontal axis F perpendicular to the sagittal plane. The first angular offset may accommodate for a vertical elevation of the zone of intraoral tenderness from the bottom of the upper molars, by a tilting of the probes. The first angular offset is selected to elevate or lower the cooling element 12. The first angular offset may be between about 0° and 15° from the transverse plane. In some embodiments the first angular offset may be about 0° and 25° from the transverse plane. In some embodiments the first angular offset may be greater than 25° though a first angular offset less than 25° seems appropriate.

The one or more angular offsets may include a second angular offset about a sagittal axis S perpendicular to the frontal plane of between about +10° and −10°. In some embodiments the second angular offset may be about −15° and 15° from the transverse plane. The second angular offset may be greater than 15° or less than −15° though a second angular offset less than 15° or greater than −15° seems appropriate. The second angular offset accommodates for variations in a lateral displacement of the zone of intraoral tenderness. The second angular offset of the bite pad 24 effectively rotates the cooling element 12 inwardly or outwardly, depending on the selected second angular offset, for proper abutment of the cooling element 12 with the zone of intraoral tenderness and so that the user remains comfortable with the hands-free intraoral device 10 throughout the treatment period. When the positioning element 16 is formed of a resilient material, the cooling element 12 may be resiliently urged in abutment with the zone of intraoral tenderness. In an exemplary embodiment, a vertex angle formed at an intersection of a longitudinal centerline of the cooling element 12 and a distal edge of the bottom of the bite pad 24 may be about 20 degrees to about 50 degrees.

The bite pad 24 and connector 22 allow for fitting the device 10 to a number of mouth sizes since the bite pad 24, when operably engaged, will naturally push the cooling element 12 into the zone of intraoral tenderness for at least 75% of mouth sizes. Even among the smallest and largest mouth sizes, the difference in reaching the zone of intraoral tenderness can be best measured in millimeters. Thus, it has been seen in initial testing that the bite pads 24 and connector 22 allow for a natural fit for a greater percentage of patients' mouths than just 75%. Accordingly, the bite pads 24 and connector 22 can allow for a natural fit for at least 75% up to 97% of patients, possibly greater. As a result, less skus would be required, possibly only one, to accommodate nearly all patients, which is desirable for manufacturing. It is achievable by considering where most mouth sizes are similarly sized such that any differences across patients can be accounted for in millimeters.

In the present example, differences in the distance from the teeth which engage the bite pads 24 during treatment and the patient's zone of intraoral tenderness can be measured in millimeters. Further, the angular offsets (AO) needed to position the cooling element 12 in abutment with the zone of intraoral tenderness when the device is inserted are also similar. Refer to Tables 1 and 2.

Molds are also possible to achieve marginally better treatment coverage and easier application. Further, not shown, the device can be affixed to a patient's head, neck, face, chin, nose, shoulders, arms, underarms, ears, or other body part to achieve marginally better treatment coverage or easier application.

As seen in TABLE 1 below, a comparison of representative first angular offsets and second angular offsets (AO) of the bite pad 24 orientation are shown versus factors of comfort, secure fit, and cooling element 12 position are presented. The factors were rated on a scale of 1 to 5, with a score of 5 being the best and a score of 1 being the worst. The table illustrates that each combination was effective in positioning at least a portion of the cooling element 12 in abutment with the zone of intraoral tenderness.

TABLE 1
Comparison of Angular Offset Configurations

| Factor | 1^{st} A O15° | 1^{st} AO 15° | 1^{st} AO 15° | 1^{st} AO 10° | 1^{st} AO 5° |
|---|---|---|---|---|---|
| 1 to 5 | 2^{nd} AO 0° | 2^{nd} AO +10° | 2^{nd} AO −10° | 2^{nd} AO 0° | 2^{nd} AO 0° |
| Comfort | 3 | 4 | 4 | 2 | 1 |
| Secure Fit | 3 | 4 | 3 | 4 | 2 |
| Tip Position | 3 | 3 | 4 | 3 | 4 |
| Notes | Bite towards | Smaller bite | Probe end | Increased | Least |
| | edge of pad. | pad area. | feels further | pressure | comfort. |
| | Drooling @ | Drooling @ | from gumline, | on | Most |
| | 4 min. | 2 min. | which is more | gumline. | gumline |
| | Jaw Soreness | Less bite | comfortable. | Less | contact |
| | @ 10 min. | force, stress | Less | drooling | |
| | Edge of lips | on jaw | drooling | | |
| | itchy @ 14 min | | | | |

As set forth in Table 2 below, the bite pads 24 and/or vertical teeth guard/guidance plate 25 can be graded so as to hug the teeth according to the anticipated size of each tooth:

TABLE 2

| | Crown | Root | Total | M-D | F-L |
|---|---|---|---|---|---|
| Maxillary | length mm | Length Mm | Length mm | mm | mm |
| Central | 11.2* | 13.0 | 23.6 | 8.6*** | 7.1 |
| Lateral | 9.8 | 13.4 | 22.5 | 6.6*** | 6.2 |
| Canine | 10.6 | 16.5**** | 26.3** | 7.6 | 8.1 |
| 1^{st} Pre | 8.6 | 13.4 | 21.5 | 7.1 | 9.2 |
| 2^{nd} Pre | 7.7 | 14.0 | 21.2 | 6.6 | 9.0 |
| 1^{st} Molar | 7.5 | 13.7 pal | 20.1 | 10.4 | 11.5 |

*longest crown
**longest tooth overall
***only two teeth wider M-D than F-L
****longest root

As described in Table 2, the M-D measurements stand for mesiodistal crown diameter, which relates to the mesio and distal surfaces of the teeth, or tooth length. The distal tooth surface refers to the back surface of the tooth, whereas the mesial tooth surface refers to the front surface of the tooth. Similarly, the F-L measurements stand for the faciolingual dimensions of the teeth, or tooth width, with the facio side being the front, or face (or cheek) side of the tooth, and the lingual side being the back, or tongue side of the tooth.

Accordingly, in an embodiment, the bite pads 24 and/or vertical teeth guard/guidance plate 25 can be graded to align with the typical size of the premolars and molar teeth. In this regard, the bite pads 24 and/or vertical teeth guard/guidance plate 25 can have a tapered width, in the F-L and M-D direction, with the bite pads 24 and/or vertical teeth guard/guidance plate 25 getting incrementally narrower going from the side positioned at the back of the mouth, or closest to the first molar, and extending toward the side positioned towards the front of the mouth, or towards the premolars. In one exemplary, non-limiting embodiment in this regard, the bite pads 24 and/or vertical teeth guard/guidance plate 25 can have an about 22 mm width at the first molar side tapering down to an about 18 mm width at the premolar side.

Similarly, the bite pads 24 and/or vertical teeth guard/guidance plate 25 can have a tapered height, with the bite pads 24 and/or vertical teeth guard/guidance plate 25 getting incrementally longer, or increasing in height, going from the side positioned at the back of the mouth, or closest to the first molar, and extending toward the side positioned towards the front of the mouth, or towards the premolars. In one exemplary, non-limiting embodiment in this regard, the bite pads 24 and/or vertical teeth guard/guidance plate 25 can have an about 10 mm height at the first molar side tapering up to an about 12 mm height at the premolar side.

In certain embodiments, the full length of the bite pads 24 and/or vertical teeth guard/guidance plate 25 extending from the front of the mouth/chin side to the back of the mouth/neck side can be maintained, by way of non-limiting example, at about 35 mm, obtained by adding the M-D measurements of the premolars, the 1^{st} molar, plus a certain extension amount, or any other suitable length. An aspect of the discovery herein is that any standard deviation in the distance from certain teeth to the zone of intraoral tenderness can be best measured in millimeters and thus makes a generalized design concept possible even though large and small mouths may physically present different challenges when it comes to reaching and appropriately treating the zone of intraoral tenderness. In conjunction, the similarly sized teeth from small patient to large patient where differences can also be measured in millimeters was known and can make the generalized designs possible. These sizing considerations will be particularly effective for the tooth clip variation functioning as a tooth wedge, but can play a fundamental role in all bite pad embodiments. The tooth wedge application can be very effective in treating unconscious patients. It, as well as other embodiments, can be equipped with something to in some way affix the device to the patient's head, neck, face, chin, nose, shoulders, arms, underarms, ears, or other body part for stabilization.

As seen in FIGS. 1-8, 31-35, 37-44, and 49-50, a vertex angle formed at an intersection of a longitudinal centerline of the cooling element 12 and a distal edge of the bottom of the bite pad 24 may be about 20 degrees to about 50 degrees. This vertex angle can be used for a multitude of patients so that the cooling element effectively contacts the zone of intraoral tenderness consistently, easily, and comfortably. In some embodiments, the vertex angle can be 20 degrees to 35 degrees.

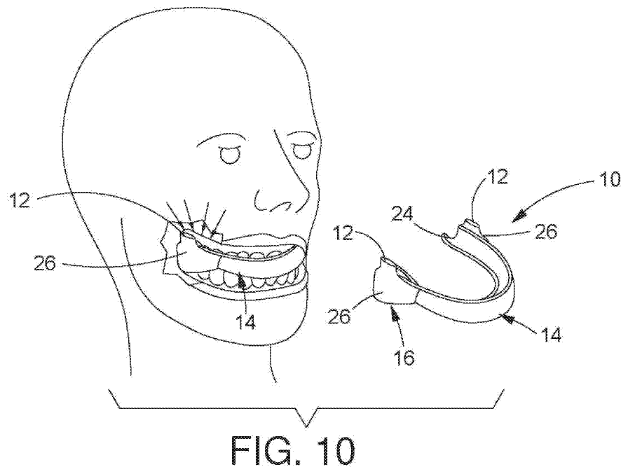

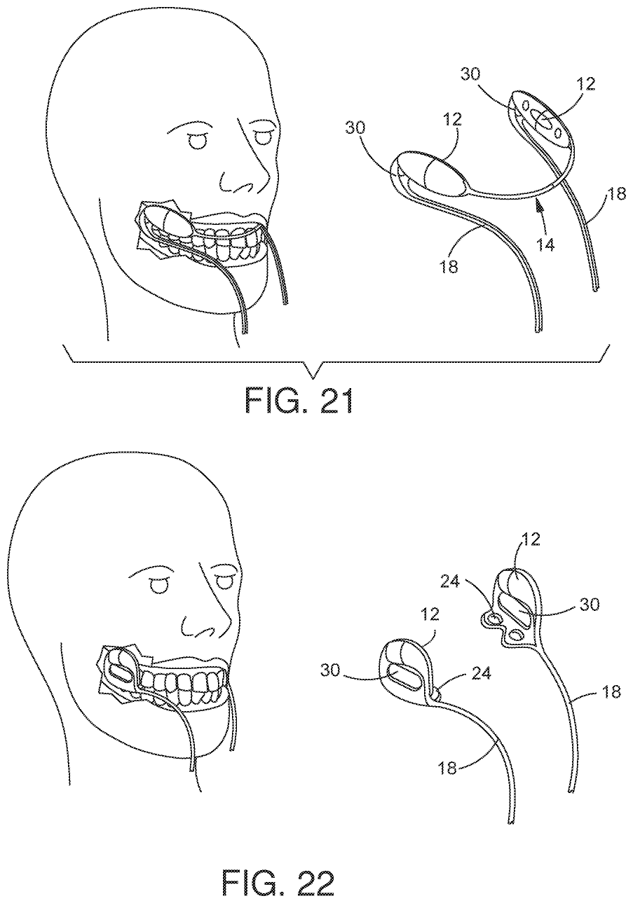

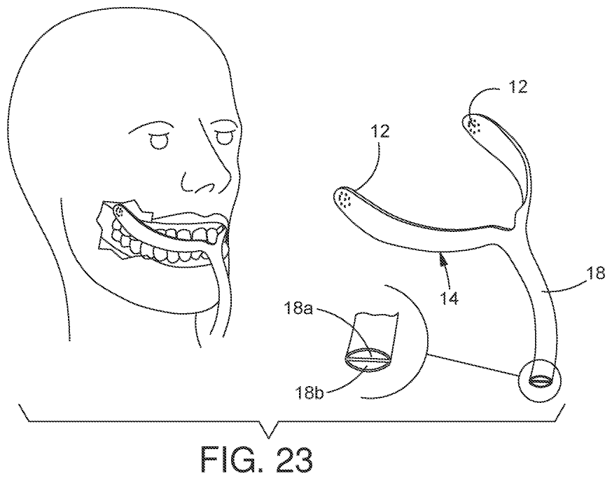

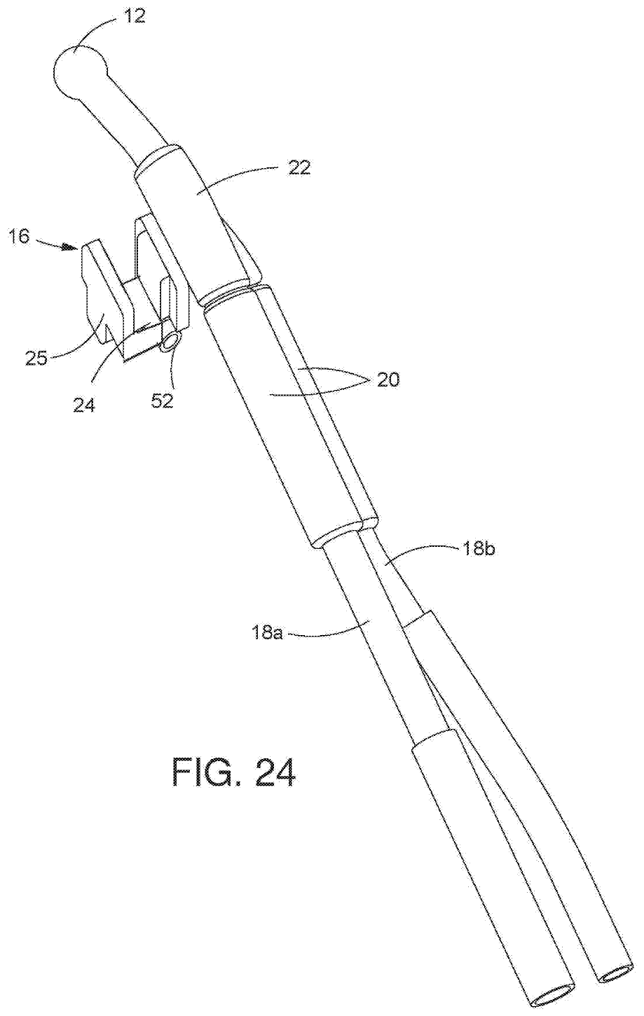

As seen in reference to FIGS. 10-23 and 36, the carrier 14 of many embodiments is formed as a generally U-shaped carrier. A distal end of the mouthpiece is dimensioned to be received within the intraoral cavity. In these embodiments, at least a portion of the U-shaped carrier 14 is positioned in the cheek fold, or pocket, defined between an upper jawline of the user and a cheek of the user. The cooling element 12 is disposed at a distal end of the U-shaped carrier 14.

In the example shown in reference to FIG. 10, the positioning element 16 may include a protrusion 26 defined at the distal end of the U-shaped carrier 14. The cooling element 12 is disposed at a top end of the protrusion 26 used for positioning of the cooling element 12 in abutment with the zone of intraoral tenderness. The protrusion 26 may also have a thickness that engages with a surface of the user's cheek to urge the cooling element 12 laterally for abutment with the zone of intraoral tenderness.

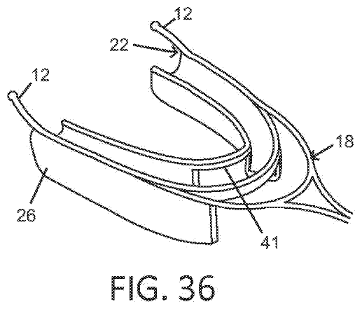

In some embodiments, such as the embodiment of FIG. 36, the hands-free intraoral device 10 can include an air flow bridge 41 to promote oxygen flow into the mouth, while in use. This embodiment can create a more relaxing experience, in use, resulting in a patient who is more likely to successfully comply with the full treatment regimen. The cooling element would sit atop an intentionally high outer guiding wall of the mouthguard that can be thought of as a protrusion 26.

In the embodiment shown in reference to FIG. 10, the carrier 14 is U-shaped formed similar to that of a mouthguard. The U-shaped carrier 14 may also include a bite pad 24 extending transversely towards a sagittal centerline from an outer side of the U-shaped carrier 14. As described previously, the bite pad 24 may be oriented at one or more angular offsets to facilitate fitting of the hands-free intraoral device 10 to position the cooling element 12 in abutment with the zone of intraoral tenderness or can be molded to fit the user's mouth. The bite pad 24 is configured to be engaged by a biting of one or more molar teeth of the user teeth to position the cooling element 12 in abutment with the zone of intraoral tenderness and to retain the hands-free device within the intraoral cavity. The conduit 18 may be carried within the U-shaped carrier 14. Alternatively, a thermoelectric cooler (TEC), such as a Peltier device, or a frozen mass may be housed within the protrusion 26. A frozen mass cooled in a separate contraption, such as a freezer, can be reasonably used for the cooling element 12 or lower cooling element 37.

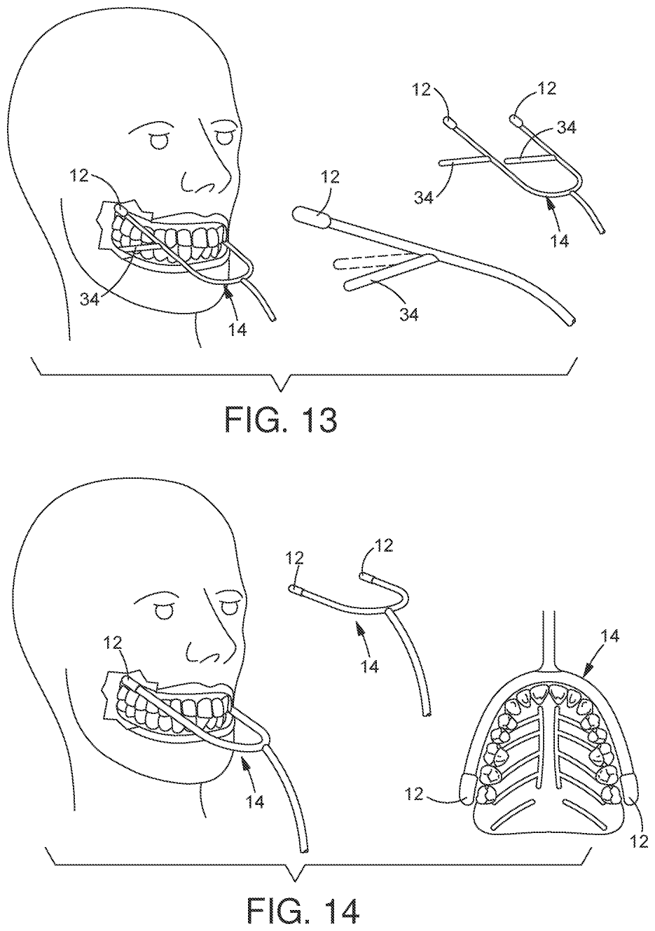

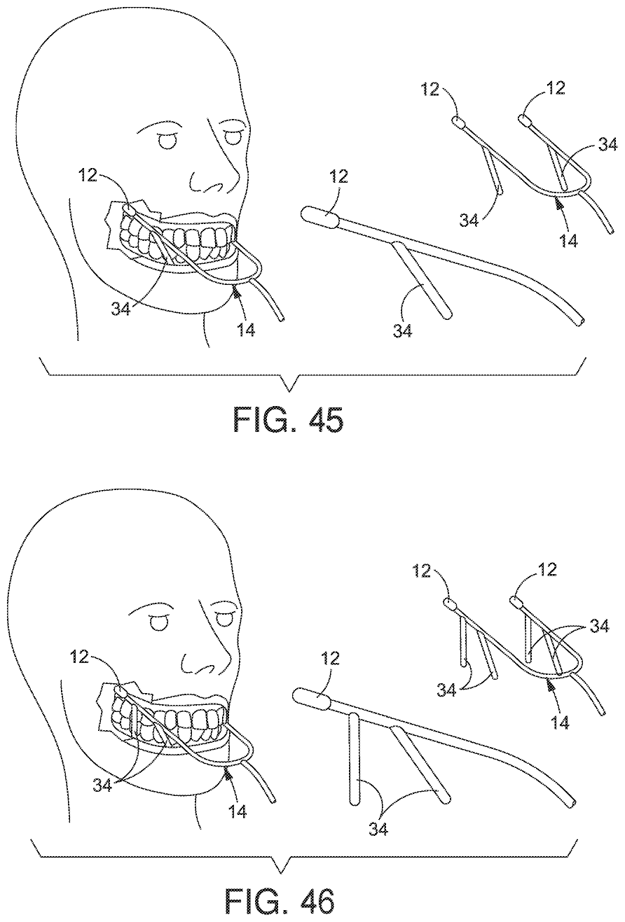

In the embodiment shown in reference to FIGS. 13 and 45-48A, at least one flexible arm 34 diverges from the carrier 14. The at least one flexible arm 34 is dimensioned to resiliently engage between the cheek fold along the jawline and the cheek of the user. The at least one flexible arm 34 is operable by the user's cheek and/or jaw to urge the cooling element 12 in abutment with the zone of intraoral tenderness, forming a multi-pronged embodiment. These embodiments are particularly useful in instances where the patient is unconscious as the hands-free intraoral device 10 requires no motor function for a hands-free operation.

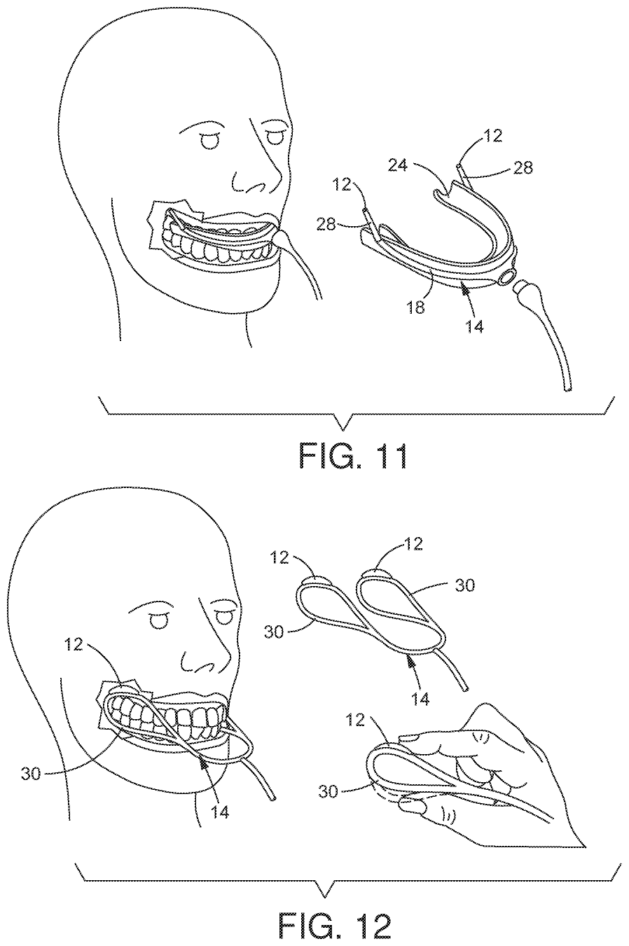

In the embodiment shown in reference to FIGS. 11, and 16-18, the cooling element 12 may be formed as a cylindrical extension 28 oriented at an elevated angular offset from the U-shaped carrier 14 about a frontal axis F, which can be a custom molded fit. As with previous embodiments, the bite pad 24 may be incorporated with the U-shaped carrier 14. In these embodiments, the cylindrical extension 28 may be formed of a resilient material to provide for a resilient urging of the cooling element 12 into abutment with the zone of intraoral tenderness.

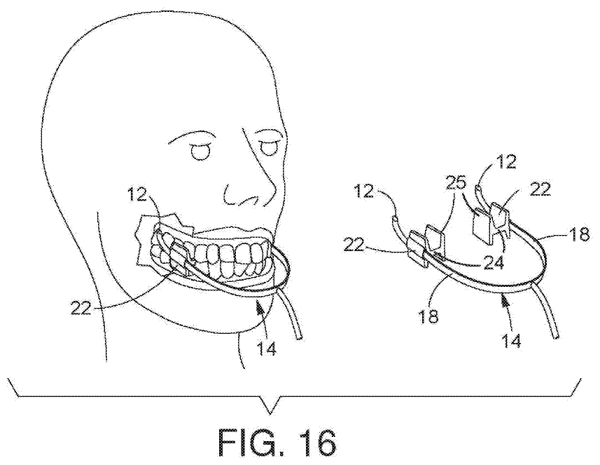

In the embodiment shown in FIGS. 16, 24-26, 31-35, 37-42, and 49-50, a vertical teeth guard/guidance plate 25 is coupled with the bite pad 24 in a vertical orientation. The vertical teeth guard/guidance plate 25 allows for easier insertion of each prong into the oral cavity. Further, it helps protect the teeth from contacting the cooling element 12. It can also be used to house hardware or other widgets to assist with the treatment.

In the embodiment shown in FIGS. 24-26, 35, and 49-49A, the positioning element 16 is a tooth clip variation of the bite pad 24. In some embodiments the vertical teeth guard/guidance plate 25, positioned at an interior and exterior end of the bite pad 24 are flexibly coupled via a hinge 52. The vertical teeth guard/guidance plate 25 and the bite pad 24 are biased towards the carrier 14 via a tensioning mechanism (not shown) to clip the distal end of the carrier 14 to an interior and exterior surface of the one or more molars of the user. The tooth clip variation can function as a tooth wedge in which the positioning element hugs the teeth and a hinge 52 may not be required.

A flexible loop 30 positioning element may be seen in reference to the drawings of FIGS. 12, and 19-22. In these embodiments, the carrier 14 may be considered a U-shaped carrier 14. The positioning element 16 is formed as a flexible loop 30 defined at the distal end of the carrier 14. The flexible loop 30 is dimensioned to be resiliently received in the cheek folds between the upper jawline, the lower jawline, and the cheeks of the user. The flexible loop 30 is operable to urge the cooling element 12 in abutment with the zone of intraoral tenderness by the user closing their lower jaw. In this case the flexible loop 30, when positioned in the cheek folds is compressed by the closure of the user's lower jaw. The cooling element 12 is disposed along an upper arc of the flexible loop 30. The flexible loop 30 may contain or be in fluid communication with the conduit 18, which may also be covered in insulation material 20. As seen in FIG. 20, a supplemental loop 31 may be provided protruding towards the user's cheek to urge the cooling element 12 inwardly towards the zone of intraoral tenderness.

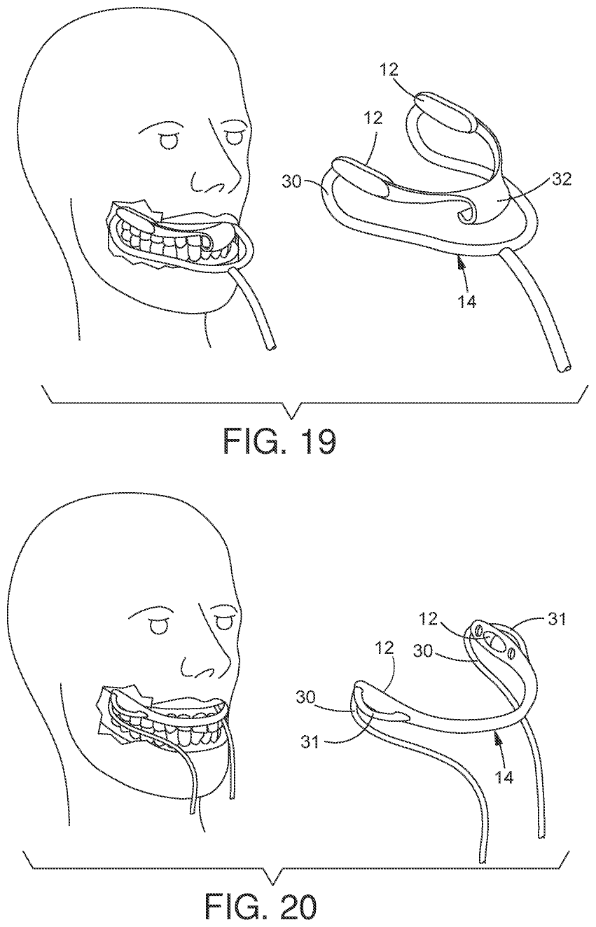

In the embodiment shown in reference to FIG. 19 a bite clip 32 is disposed at a forward end of the flexible loop 30. The bite clip 32 is configured with a lip to engage with the user's incisors and facilitates positioning and retention of the hands-free intraoral device 10 in the intraoral cavity.

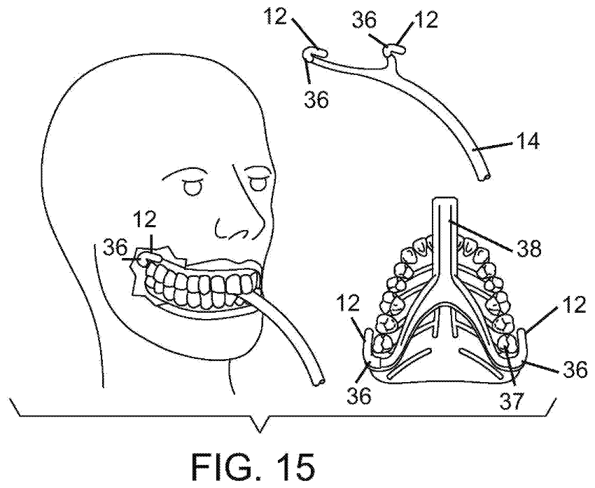

In the embodiment shown in reference to FIG. 15, the positioning element is formed as a hook 36 defined at the distal end of a wishbone shaped carrier 38. In this embodiment, the wishbone shaped carrier 38 is dimensioned to be contained or positioned in the oral pallet. The hook 36 is dimensioned for engagement around a distal end of the maxillary arch 62 of the user. The cooling element 12 is positioned at a terminal end of the hook 36 and positioned for abutment with the zone of intraoral tenderness. For sizing of this embodiment, the terminal end of the hook 36 is disposed at an elevated angle relative to a transverse plane of the intraoral cavity. The terminal end of the hook 36 may also be formed as a resilient element for resiliently urging the cooling element 12 in abutment with the zone of intraoral tenderness.

In some embodiments, a cooling medium (not shown) is in communication with the conduit 18. The cooling medium is configured to circulate the cooling fluid through the conduit 18 of hands-free intraoral device 10 and may be used to provide the cooling of the cooling element 12.

A much more relaxing experience from a breathability and a comfort perspective is achieved with the open front mouth designs which the bite pads 24 allow for, increasing the likelihood of treatment compliance. Patients can hold conversations with the device in their mouth. Perhaps most importantly, patients no longer have to worry about the probes moving out of the treatment area with a slight movement of their hand, wrist arm, or mouth, which can routinely happen via fatigue, human error, or practitioner error without a positioning element 16.

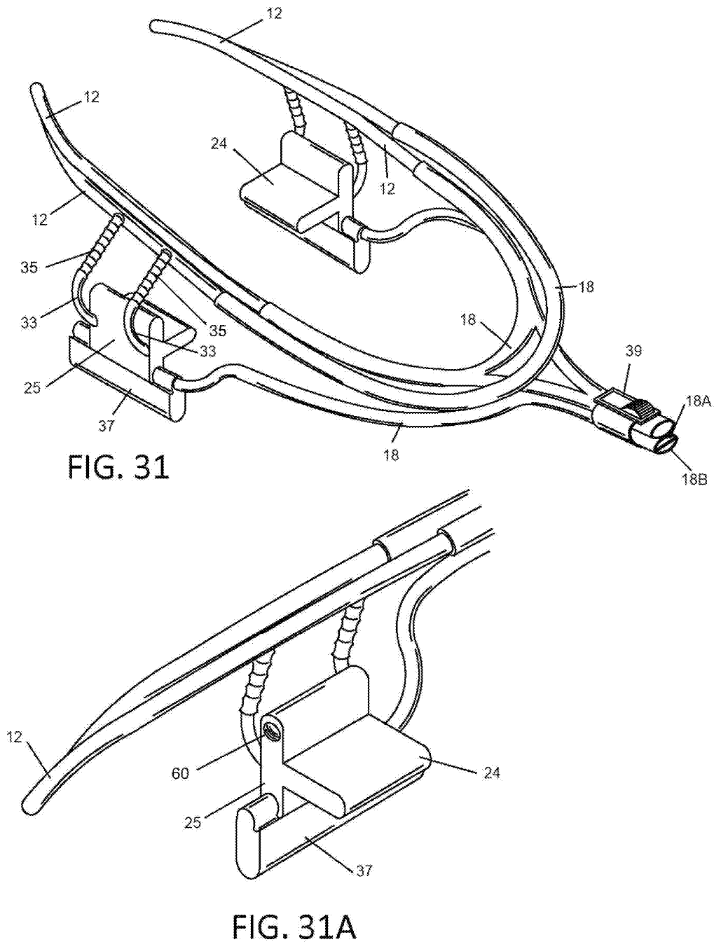

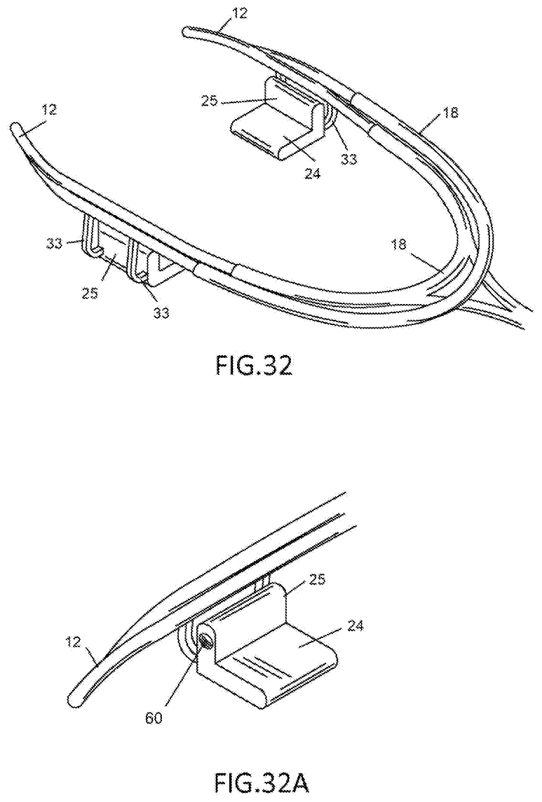

In some embodiments, the hands-free intraoral device 10 has a vertical teeth guard/guidance plate 25 that includes a diode, helium-neon, or other laser 60 as shown in FIGS. 31A and 32A. According to this embodiment, the vertical teeth guard/guidance plate 25 has a small circular window which a laser 60 emits out of. The window, or aperture, can be tilted slightly upwards towards the maxillary, or upper, gumline area, or downwards towards the mandibular, or lower, gumline using a lever (not shown). In this embodiment, the vertical teeth guard/guidance plate 25 houses the laser 60, which can shoot pulses intermittently throughout treatment or by manipulating a lever (not shown) to activate the laser 60. This laser 60 can help improve blood flow in the area to which it is directed and can improve the effectiveness of the treatment. A communications module 46 (not shown) can also be used to activate or deactivate the laser 60 throughout the treatment from a mobile computing device 48 (not shown) among other tasks aiding in treatment.

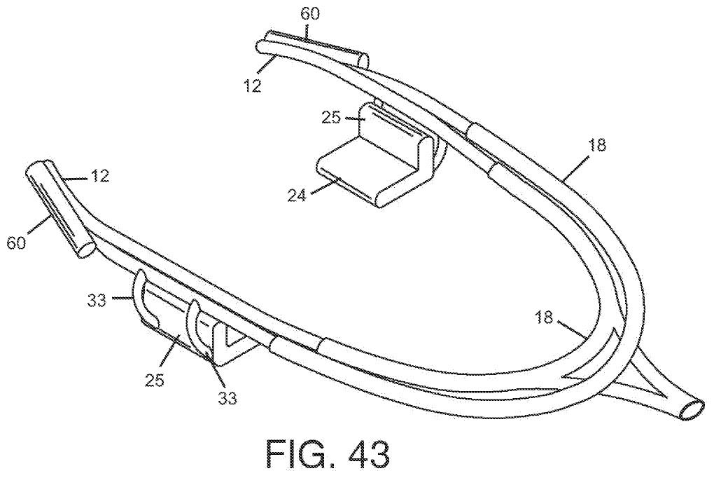

In an alternative embodiment, as shown in FIG. 43, a diode laser 60 can be included in a discrete unit separate from the vertical teeth guard/guidance plate 25. In the non-limiting embodiment of FIG. 43, a unit housing the diode laser 60 can be attached to the cooling element 12.

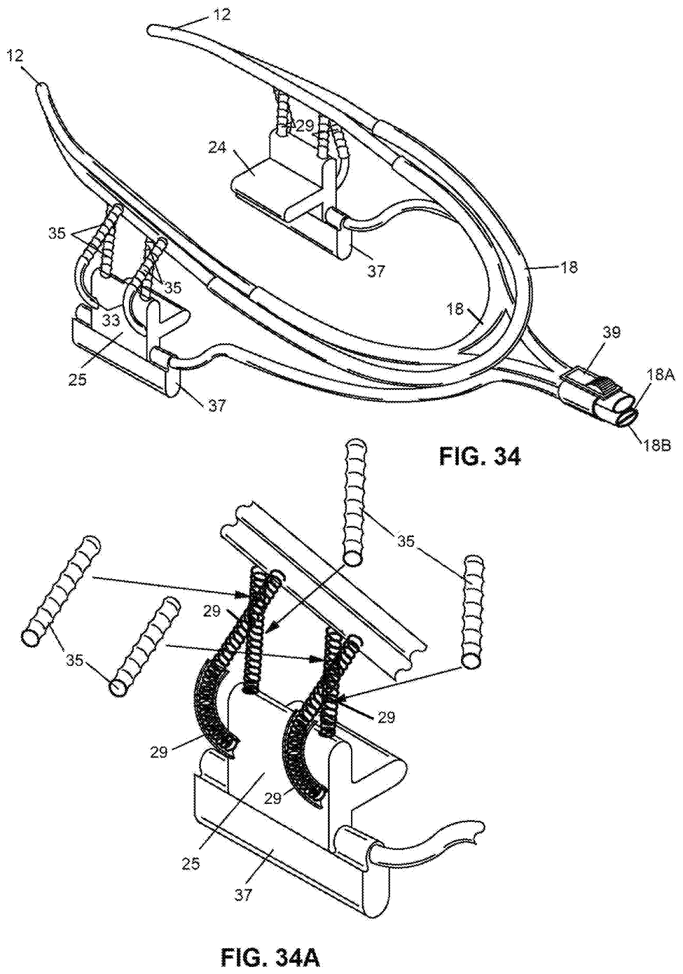

As shown in FIGS. 31, 33-34, 41-42, 47-48A, and 49-50, another embodiment includes a hooked support and flexible attachment material 29, each acting as a connector 22 linking the positioning element 16 to the cooling element 12. The flexible attachment material 29 can, by way of non-limiting example, be a spring, a suitably flexible material, or contraption which is flexible and can be compressed, expanded, or forced into position. In the case where the flexible attachment material 29 is a spring, the spring can be encased in a sleeve 35 of a flexible comfortable material which can expand or contract with the flexible attachment material 29. The flexible attachment material 29 can be used to pressure the cooling element 12 up and inward simultaneously when the hands-free intraoral device 10 is in the mouth. In another embodiment, as shown in FIG. 34, there can be multiple flexible attachment materials 29 connecting the positioning element 16 at both an outer edge and a top edge to each cooling element 12. The number of flexible attachments necessary is not limited and can extend from any edge of the cooling element 12 or positioning element 16 to ensure a proper fit.

In certain embodiments, as shown in FIGS. 31 and 33-34, a switch 39 can be placed on the hands-free intraoral device 10 to move the flexible attachment material 29 up and inward or down and outward as desired to allow for a more comfortable insertion of the carrier 14 into the oral cavity as well as a tighter fit during treatment. Using the switch 39 in this embodiment, the flexible attachment material 29 can be moved either to an up and inward position or a down and outward position, or can be moved to varying points in between each of the up and inward or down and outward positions. The switch 39 can include a locking mechanism to lock the flexible attachment material 29 in place once a desired position has been reached. Though the bite pad 24 positioning element 16 seen in FIGS. 1-8 and FIG. 44 can fit a normalized distribution of mouth sizes greater than 75%, and in some embodiments greater than 95%, the switch 39 may allow for an even better fit.

In other embodiments such as in FIGS. 31-35, 37-43, 47-48A, and 49-50, the hands-free intraoral device 10 can include a connector 22 in the form of a set of supports 33. These supports 33 can be resilient, semi-rigid, or rigid. They may permit more optimal positioning of the cooling element and provide comfort by allowing for a sleeker design. A sleeker design is desirable given the zone of intraoral tenderness is towards the back of the mouth and near the throat so there is not much room for additional parts during the duration of the treatment. Additionally, the supports 33 can be used in combination with the flexible attachment material 29 to point the flexible attachment material 29 to a particular angle so that the cooling element 12 may be optimally positioned. These embodiments can rely on rounded edge and hooked supports, however, supports 33 can come in a variety of shapes and rigid, semi-rigid, or resilient materials. The flexible attachment material 29 can be housed in the supports 33.

In embodiments shown in FIGS. 31, 33-34, 38, 40-42, and 47-48A, the hands-free intraoral device 10 can include a lower cooling element 37 to cool the mandibular gumline and zone of intraoral tenderness simultaneously. It is often the case that temporomandibular joint disorder patients are susceptible to migraine. The lower (cooling) element 37 may be used to aid in positioning, comfort, and need not be used for chilling.

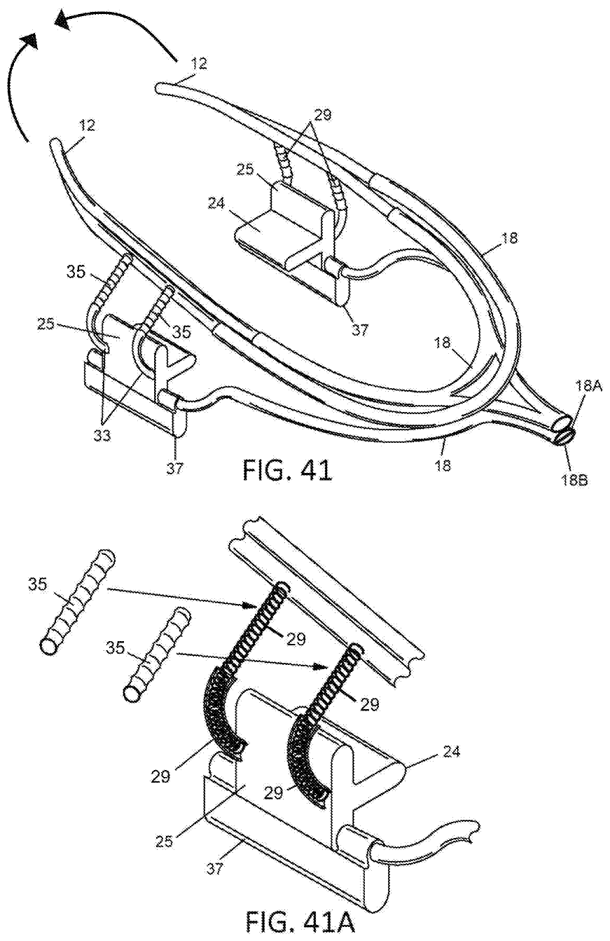

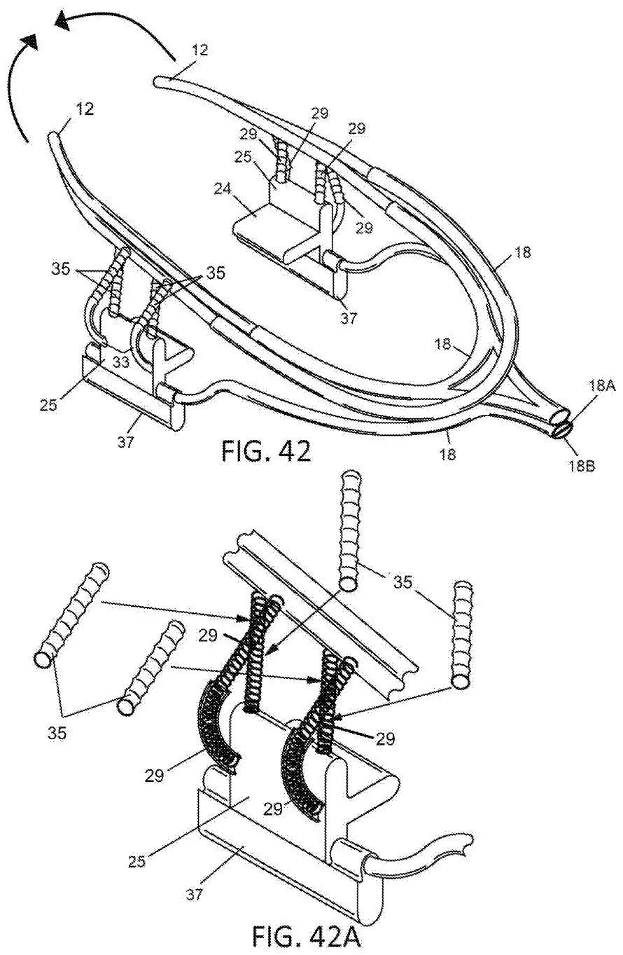

In FIGS. 41-42, the cooling element is tensed inward towards the tongue and upwards towards the roof of the mouth by a tensioning force of the connector 22 comprising the flexible attachment material 29, in this case a spring, which is covered by a sleeve 35, and supports 33 angled to point the flexible attachment materials 29 in the appropriate direction. Once inserted in the oral cavity, one prong at a time, the user can manually adjust the cooling element 16 to outside the gums using their fingers and hands and then bite down on the positioning element 16, in this case bite pads 24. When biting on the bite pads 24, the spring flexible attachment material 29 tenses the cooling element 12 in abutment with the zone of intraoral tenderness. The tension of the spring flexible attachment material 29, which is guided by the supports 33, will force the cooling element 12 upwards and inwards allowing it to abut the zone of intraoral tenderness and chill the correct location. The flexible attachment material 29 can be as numerous as necessary and angled in a multitude of directions to ensure abutment with the zone of intraoral tenderness.

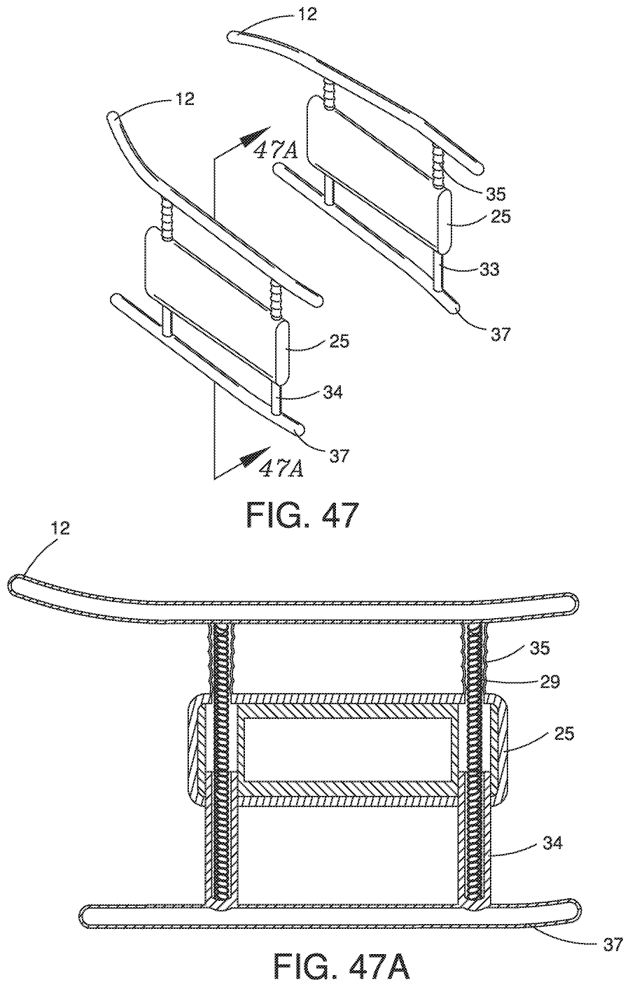

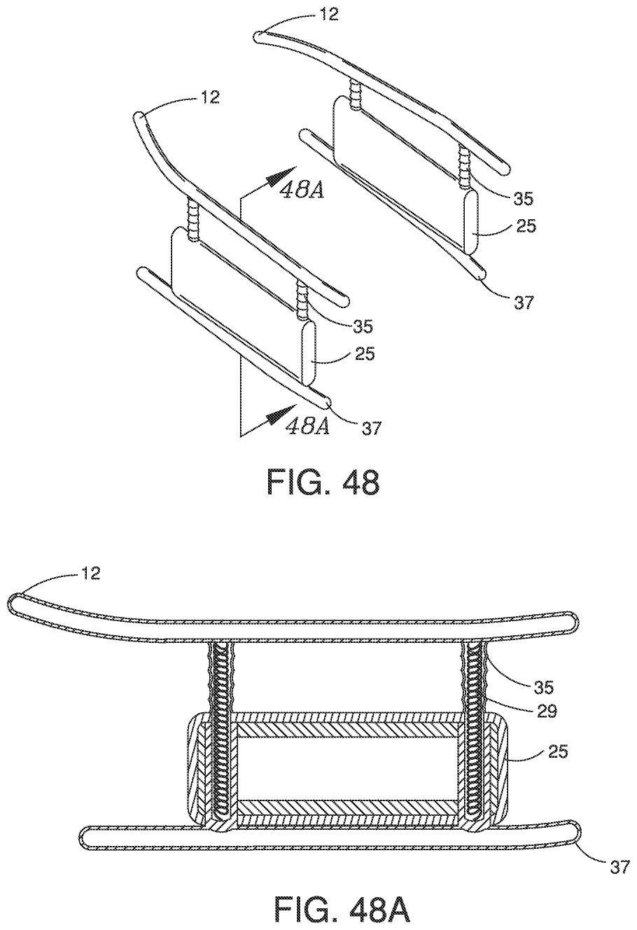

In some embodiments, as shown in FIGS. 47-48A, the hands-free intraoral device 10 can have a vertical teeth guard/guidance plate 25 without a bite pad 24. According to these embodiments, a flexible arm 34 in the form of flexible attachment material 29 housed in a set of supports 33 or the vertical teeth guard/guidance plate 25 is used to position the cooling element 12 in a patient's cheek, resiliently engaging against the lower jaw to stabilize and direct the cooling element 12. These embodiments may include a lower (cooling) element 37. The lower (cooling) element 37 may aid in positioning, provide comfort, chilling the mandibular gumline, or a combination thereof. A button 58 (not shown) can be used to lower the cooling element 12 for easy insertion and removal.

The hands-free intraoral device 10 may also be described as imparting a heat transfer from the zone of intraoral tenderness within an oral cavity of a user. A heat exchanger 40 may be configured to transfer heat from the cooling element 12. The heat exchanger 40 may include the previously described cooling fluid circulated through the conduits 18 via the cooling medium, a thermoelectric cooler, or a thermally conductive material in contact with the vascularization of the cheek or tongue. A heat sink may be used as well and positioned under the tongue or in the upper or lower cheek fold.

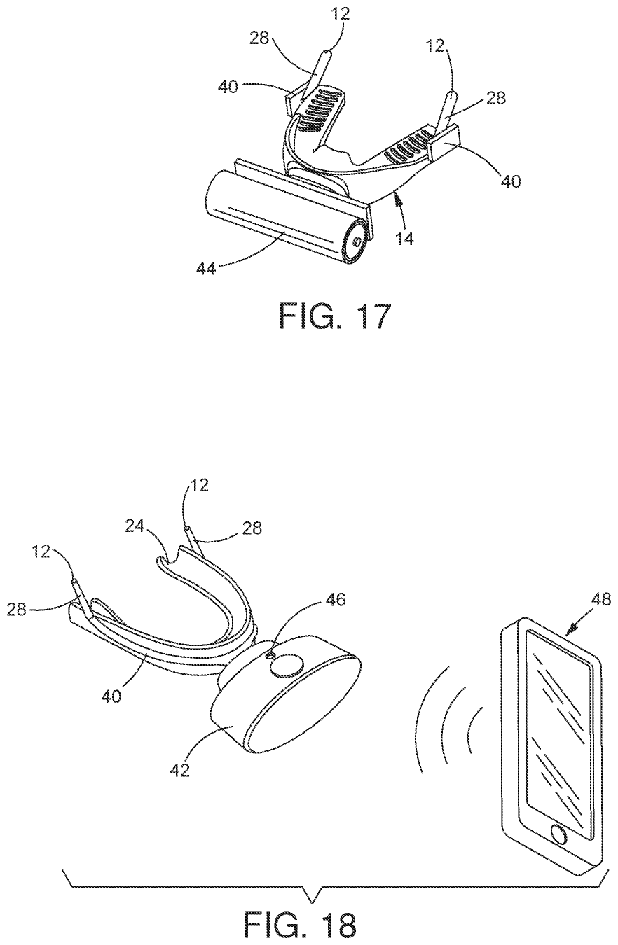

As shown in reference to FIGS. 17, 18, and 27, the heat exchanger 40 may be a component of the thermoelectric cooler thermally coupled with the cooling element 12. In this embodiment, an electronics package 42 is provided for control of the thermoelectric cooler. The electronics package 42 may include a battery 44 to power the thermoelectric cooler. In the embodiments shown, the electronics package 42 is coupled with the distal end of the carrier 14. The electronics package 42 may include a communications module 46, such as Wi-Fi, Bluetooth®, or near field communication, configured for wireless communication with a mobile computing device 48 for controlling the thermoelectric cooler.

The mobile computing device 48 may include an application for sensing and controlling the temperature at the cooling element 12 and the zone of intraoral tenderness. The application may also be configurable by the user for setting a temporal duration of the treatment session via a timer. Data, such as the temperature, duration, date, and user information may be stored in a memory of the mobile computing device 48 as a record of the treatments.

The application may also include a user interface for the user to score the subjective effectiveness of the treatments. The user interface may also allow the user to enter incidents or events that may occur between treatments. Likewise, the user interface may allow the user to enter positioning parameters for each treatment and optionally, a comfort assessment of the hands-free intraoral device 10 and the positioning of the cooling element 12 of the hands-free intraoral device 10 parameters for the treatment session. The data may be analyzed to present one or more trends for the patient's progress or to suggest or recommend one or more positioning parameter adjustments for a subsequent treatment.

The application and the mobile computing device 48 may also communicate the data, with a server, where the data may be retained as a backup for the user. The server may also be configured to analyze data from a plurality of users. An artificial intelligence (AI) module may assess the data to provide insights into treatment and/or device parameters to enhance the effectiveness of the hands-free intraoral device 10 treatment across a broad population. As will be appreciated, the electronics package 42, associated electronics component and application may be utilized with one or more embodiments disclosed herein.

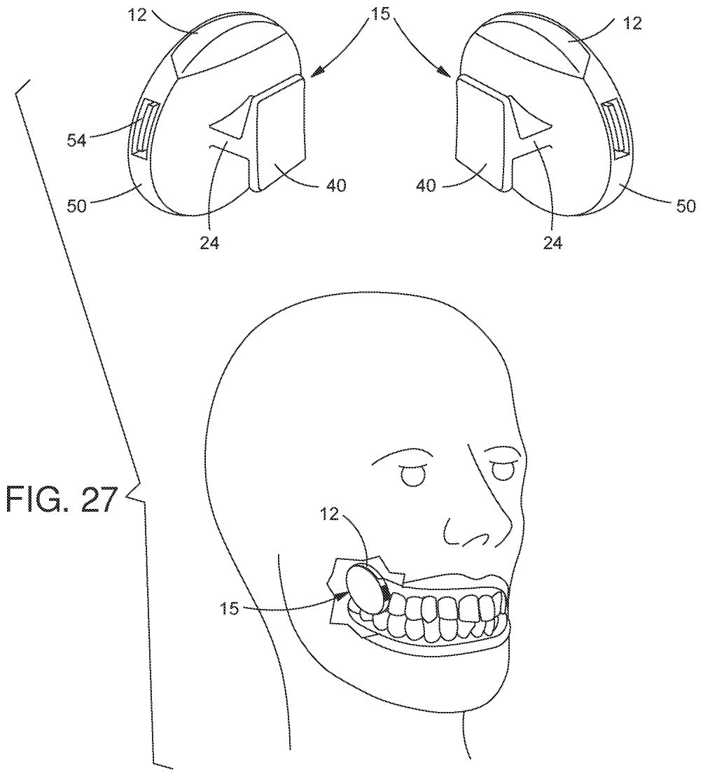

A bite knob embodiment of the hands-free intraoral device 10 is illustrated in reference to FIG. 27. In this embodiment, the carrier includes a disc shaped housing 50 having an inner face and an outer face. The disc shaped housing 50 is configured to contain a TEC, such as the Peltier device. The cooling element 12 is positioned at an upper arch of the disc shaped housing 50, and more preferably extends along an upper margin of the inner face of the disc shaped housing 50 for improved positioning with the zone of intraoral tenderness.

A bite pad 24 extends transversely from the inner face of the disc shaped housing 50 at a connector 22 and is operable by the molars of the user to position the cooling element 12 in abutment with the zone of intraoral tenderness. In this embodiment, a heat exchanger 40 is disposed at an interior end of the bite pad 24. The heat exchanger 40 is configured to communicate heat from the TEC to be dissipated by the vascularization of the user's tongue. The TEC may be powered by a battery source carried within the disc shaped housing 50, which may be charged via a charging port-54. Alternatively, an external power supply may be attached to the charging port 54.

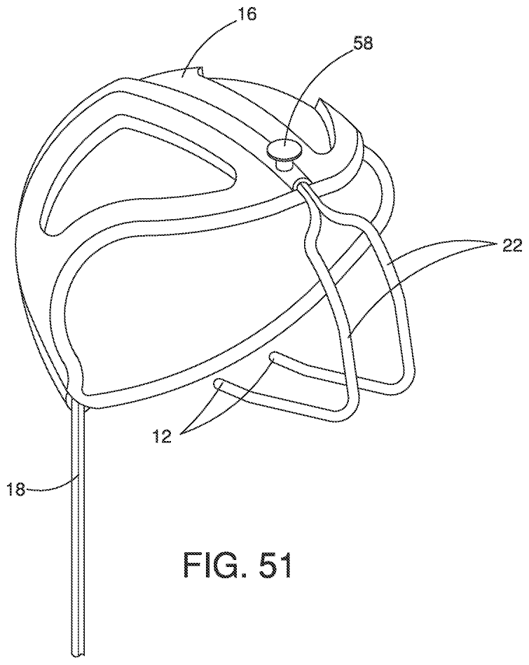

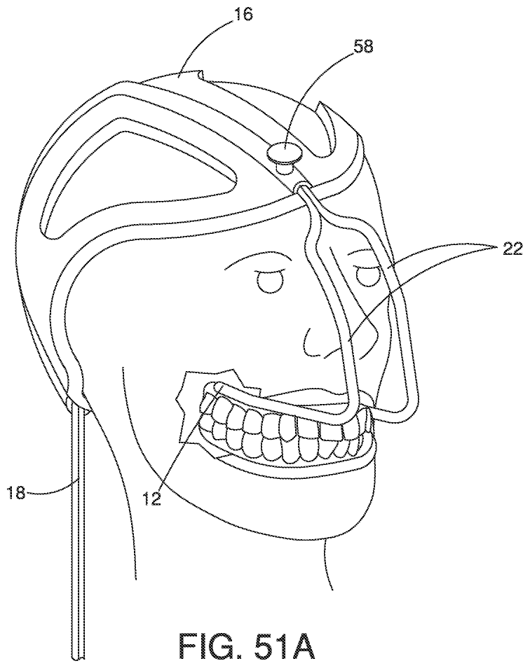

A clamping embodiment of the hands-free intraoral device 10 may be seen in reference to FIGS. 29 and 51-51A. In this embodiment, the hands-free intraoral device 10 also includes the cooling element 12 disposed at the distal end of the carrier 14. In this embodiment, the left and the right arms of the carrier 14 are releasably biased towards a sagittal plane oriented along a centerline of the intraoral cavity, such that the cooling element 12 is urged in abutment with the zone of intraoral tenderness. The left and the right arms of the carrier 14 may be flexible. A hinge 56 may also couple the left and the right arms of the carrier 14.

In this embodiment, the positioning element 16 includes a tensioning mechanism at a proximal end of the carrier 14, that is operable via a button 58 on one or opposed ends of the positioning element 16. In the embodiment shown, a pressing of the buttons 58 operably urges the left and the right arms of the carrier 14 outwardly, whereas a releasing of the button 58 urges the left and right arms of the carrier 14 inwardly to urge the cooling element 12 in abutment with the zone of intraoral tenderness. In this embodiment, the positioning element 16 may be carried externally of the intraoral cavity.

It should be understood, of course, that the foregoing relates to exemplary embodiments of the invention and that modifications may be made without departing from the spirit and scope of the invention as set forth in the following claims.

1. A hands-free intraoral device for imparting a chilling to a zone of intraoral tenderness within an intraoral cavity of a user, the zone of intraoral tenderness defined by an area of a plexus formed by one or more of a posterior superior and a middle superior alveolar branch of an ipsilateral maxillary nerve, the hands-free intraoral device comprising:
a carrier having a proximal end and a distal end, the distal end configured to be received within the intraoral cavity;
a cooling element disposed at the distal end of the carrier, the cooling element formed of a thermally conductive material selected to impart the chilling to the zone of intraoral tenderness; and
a positioning element to position at least a portion of the cooling element in abutment with the zone of intraoral tenderness, wherein the positioning element provides for a hands-free positioning of the cooling element in abutment with the zone of intraoral tenderness.

2. The hands-free intraoral device of claim 1, the carrier further comprising:
a connector positioned proximally to the positioning element, the connector acting as a point from which the cooling element and the positioning element separately extend.

3. The hands-free intraoral device of claim 2, the carrier further comprising:
a left prong and a right prong, each of the left prong and the right prong dimensioned to extend the cooling element to contact the zone of intraoral tenderness, when inserted in the intraoral cavity.

4. The hands-free intraoral device of claim 3, the positioning element further comprising:
a bite pad extending from the connector towards a sagittal centerline of the intraoral cavity, the bite pad configured to be operably engaged by a biting of one or more molar teeth of a user to position the cooling element in abutment with the zone of intraoral tenderness and to retain the hands-free intraoral device within the intraoral cavity.

5. The hands-free intraoral device of claim 4, wherein the bite pad has a first angular offset about a frontal axis of between about 0° and 25° from the transverse plane.

6. The hands-free intraoral device of claim 4, wherein the bite pad has a first angular offset about a frontal axis of about 5° and 15° from the transverse plane.

7. The hands-free intraoral device of claim 4, wherein the bite pad has a second angular offset about a sagittal axis of between about −15° and +15° from the transverse plane.

8. The hands-free intraoral device of claim 4, wherein the bite pad has a second angular offset of about −10° and +10°.

9. The hands-free intraoral device of claim 4, further comprising:
a vertical teeth guard/guidance plate positioned at an interior and an exterior end of the bite pad to form a bite pad-vertical teeth guard/guidance plate contraption.

10. The hands-free intraoral device of claim 9, further comprising:
a tensioning mechanism operable to bias the guidance plate against an interior and an exterior surface of the one or more molar teeth of the user.

11. The hands-free intraoral device of claim 1, the carrier further comprising:
a U-shaped carrier dimensioned to be received within the intraoral cavity, wherein at least a portion of the U-shaped carrier is positioned between an upper jawline of the user and a cheek of the user; and
the cooling element is disposed at a distal end of the U-shaped carrier.

12. The hands-free intraoral device of claim 11, the positioning element further comprising:
a bite pad extending along a distal end of the U-shaped carrier towards a sagittal centerline of the intraoral cavity, the bite pad configured to be operably engaged by a biting of one or more molar teeth of a user to position the cooling element in abutment with the zone of intraoral tenderness and to retain the hands-free intraoral device within the intraoral cavity.

13. The hands-free intraoral device of claim 11, further comprising:
a cylindrical extension oriented at an elevated angular offset from the U-shaped carrier about a frontal axis; and
the cooling element disposed at a terminal end of the cylindrical extension.

14. The hands-free intraoral device of claim 1, the positioning element further comprising:
a protrusion defined at the distal end of the carrier; and
the cooling element disposed at a top end of the protrusion.

15. The hands-free intraoral device of claim 1, the positioning element comprising:
means to flexibly urge the cooling element in abutment with the zone of intraoral tenderness.

16. The hands-free intraoral device of claim 1, the positioning element further comprising:
a flexible loop defined at the distal end of the carrier, the flexible loop dimensioned to be resiliently received between an upper jawline, a lower jawline, and a cheek of the user; and
the cooling element is disposed along an upper arc of the flexible loop.

17. The hands-free intraoral device of claim 1, the positioning element further comprising:
at least one flexible arm diverging from the carrier, the at least one flexible arm configured to resiliently engage between a jawline and a cheek of the user to position the cooling element in abutment with the zone of intraoral tenderness.

18. The hands-free intraoral device of claim 1, the positioning element further comprising:
a hook defined at the distal end of the carrier, the hook dimensioned for engagement around a distal end of the maxillary arch of the user; and
the cooling element is positioned at a terminal end of the hook.

19. The hands-free intraoral device of claim 18, wherein the terminal end of the hook is disposed at an elevated angle relative to a transverse plane of the intraoral cavity.

20. The hands-free intraoral device of claim 1, the positioning element further comprising:
a tensioning mechanism at a proximal end of the carrier, the tensioning mechanism biasing a left arm and a right arm of the carrier towards a sagittal plane oriented along a centerline of the intraoral cavity, such that the cooling element is urged in abutment with the zone of intraoral tenderness.

21. A hands-free intraoral device for imparting a heat transfer from a zone of intraoral tenderness within an intraoral cavity of a user, the zone of intraoral tenderness defined by an area of a plexus formed by one or more of a posterior superior and a middle superior alveolar branch of an ipsilateral maxillary nerve, the hands-free intraoral device comprising:
a carrier having a proximal end and a distal end, the distal end configured to be received within the intraoral cavity;
a cooling element disposed at the distal end of the carrier, the cooling element formed of a thermally conductive material selected to impart the heat transfer from the zone of intraoral tenderness to the cooling element;
a heat exchanger configured to transfer heat from the cooling element; and
a positioning element operable to position at least a portion of the cooling element in abutment with the zone of intraoral tenderness, wherein the positioning element provides for a hands-free positioning of the cooling element in abutment with the zone of intraoral tenderness.

22. The hands-free intraoral device of claim 21, the heat exchanger comprising:
a thermoelectric cooler coupled with the cooling element.

23. The hands-free intraoral device of claim 22, further comprising:
an electronics package for control of the thermoelectric cooler is coupled with the carrier.

24. The hands-free intraoral device of claim 23, the electronics package further comprising:
a communications module configured for a wireless communication with a mobile computing device for controlling the thermoelectric cooler.

25. The hands-free intraoral device of claim 1, further comprising:
a conduit to communicate a cooling fluid through the hands-free intraoral device to the cooling element;
a cooling medium in communication with the conduit, the cooling medium configured to chill the cooling element of the hands-free intraoral device; and
optionally a lower cooling element to provide further cooling to a mandibular gum line.

26. The hands-free intraoral device of claim 9, wherein the guidance plate further comprises a diode, helium-neon, or other laser and an aperture out of which the laser can shoot pulses intermittently throughout treatment.

27. The hands-free intraoral device of claim 3, wherein the positioning element is connected to the cooling element via a guidance plate and/or the connector, the connector comprising a flexible attachment material, the flexible attachment material comprising at least one spring or a suitable material which is flexible and can be compressed, expanded, or forced into position.

28. The hands-free intraoral device of claim 27, further comprising a tensioning force from the connector that urges the cooling element up and inwards and can be manually adjusted outside of the maxillary teeth, tensing the cooling element into abutment with the zone of intraoral tenderness when the bite pads are operably engaged in the oral cavity.

29. The hands-free intraoral device of claim 27, further comprising a switch on the hands-free intraoral device which, when engaged, can move the flexible attachment material to an up and inward position or a down and outward position, as well as to varying points therebetween; and a lock for holding the switch and thus the cooling element in position once activated.

30. The hands-free intraoral device of claim 10, further comprising a tooth wedge attachment to affix the hands-free intraoral device to teeth of a user, in use.

31. The hands-free intraoral device of claim 8, wherein the connector further comprises hook supports connecting the cooling element to the positioning element via an outside surface of the positioning element or the vertical teeth guard/guidance plate.

32. The hands-free intraoral device of claim 1, further comprising a mouth guard having an open front just below the incisors to promote air flow into the mouth, in use.

33. The hands-free intraoral device of claim 30, wherein the tooth attachment has a length of about 35 mm and is tapered, or graded, such that the bite pad-vertical teeth guard/guidance plate contraption has a width of about 22 mm at its most distal point and about 18 mm at its proximal point, a height of about 10 mm at its most distal point and about 12 mm at its proximal height and, in use, permits the tooth wedge attachment to extend across certain teeth, such as the pre molars and molars, allowing for the positioning element and cooling element to form a reasonable angle so that the cooling element may abut the zone of intraoral tenderness when the tooth wedge is engaged.

34. The hands-free intraoral device of claim 4, wherein a vertex angle formed at an intersection of a longitudinal centerline of the cooling element and a distal edge of the bottom of the bite pad is about 20° to about 50°.

35. The hands-free intraoral device of claim 4, wherein a vertex angle formed at an intersection of a longitudinal centerline of the cooling element and a distal edge of the bottom of the bite pad is about 20° to about 35°.

36. The hands-free intraoral device of claim 17, the hands-free device comprising:
a lower (cooling) element aligned along the mandibular gumline; the lower (cooling) element engaging with the gumline and cheek exclusively or in some combination for comfort.

37. The hands-free intraoral device of claim 36, the lower (cooling) element comprising a means of receiving cooling fluid via a conduit to produce a chilling effect in contact with the lower cheek or mandibular gum area.

38. The hands-free intraoral device of claim 37, the lower cooling element comprising a frozen mass that is able to be chilled in a cold environment and then affixed to the intraoral device when ready for treatment.
